(19) United States
(12) Patent Application Publication
(10) Pub. No.: US 2025/0332110 A1
(43) Pub. Date: Oct. 30, 2025

(54) PHARMACEUTICAL FORMULATIONS OF AN ANDROGEN RECEPTOR-TARGETING PROTEIN DEGRADER

(71) Applicants: Arvinas Operations, Inc., New Haven, CT (US); Novartis AG, Basel (CH)

(72) Inventors: Royal J. HASKELL, III, Durham, CT (US); Xiaoming CHEN, Westfield, NJ (US); Venkata Ramakrishna LINGAMANENI, Hamden, CT (US); Narasimha Murti VEMURI, Flemington, NJ (US); Sandro PAGANI, Basel (CH); Justus Johann LANGE, Basel (CH); Tiago PORFIRIO FONSECA, Basel (CH); Sebastien CHABAUD, Basel (CH); Philippe Michel Rene BOUILLOT, Basel (CH)

(21) Appl. No.: 19/188,966

(22) Filed: Apr. 24, 2025

Related U.S. Application Data
(60) Provisional application No. 63/638,900, filed on Apr. 25, 2024.

(51) Int. Cl. A61K 9/20; A61K 9/00; A61K 9/28; A61K 31/496

(52) U.S. Cl. CPC A61K 9/2077; A61K 9/0053; A61K 9/2009; A61K 9/2018; A61K 9/2027; A61K 9/2054; A61K 9/2813; A61K 9/284; A61K 9/2853; A61K 31/496

(57) ABSTRACT

The present disclosure relates to formulations of Compound A:

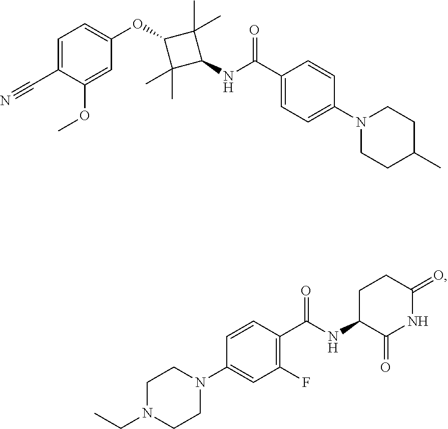

or a pharmaceutically acceptable salt thereof. The present disclosure also relates to methods of manufacturing such formulations and uses of those formulations in treating prostate cancer.

### CROSS-REFERENCE TO RELATED APPLICATIONS

This application claims priority to U.S. Provisional Application Ser. No. 63/638,900, filed Apr. 25, 2024, which is hereby incorporated herein by reference in its entirety.

### BACKGROUND

Certain bifunctional compounds are capable of targeting specific cellular proteins for degradation via the ubiquitin-proteasome system. Examples of such proteolysis targeting chimeric compounds (i.e., “PROTAC® protein degraders”) that target the Androgen Receptor (AR) for ubiquitination and subsequent degradation are disclosed in, e.g., U.S. Pat. No. 11,883,393, and U.S. application Ser. Nos. 18/460,063, 18/493,773, 18/622,152, and 63/562,672, which are incorporated herein by reference in their entireties for all purposes. Such bifunctional molecules exhibit a range of pharmacological activities consistent with the degradation of the AR including, but not limited to, treatment or amelioration of a disease condition such as cancer, including prostate cancer (e.g., metastatic prostate cancer, castrate-resistant prostate cancer, metastatic castrate-resistant prostate cancer, castrate-sensitive prostate cancer, metastatic castrate-sensitive prostate cancer).

There is a need in the art to provide improved pharmaceutical formulations for such bifunctional compounds.

### SUMMARY

A bifunctional compound of particular interest is “Compound A”, i.e.: 4-(4-((1-(4-(((1R,3R)-3-(4-cyano-3-methoxyphenoxy)-2,2,4,4-tetramethylcyclobutyl)carbamoyl)phenyl)piperidin-4-yl)methyl)piperazin-1-yl)-N-((S)-2,6-dioxopiperidin-3-yl)-2-fluorobenzamide, which has the following structure:

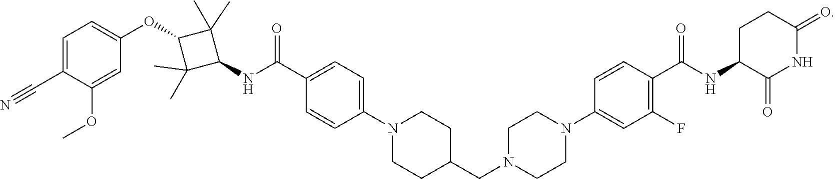 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

Compound A is under development as a PROTAC® protein degrader that targets androgen receptor (AR) for the potential treatment of prostate cancer (e.g., metastatic prostate cancer, castrate-resistant prostate cancer, metastatic castrate-resistant prostate cancer, castrate-sensitive prostate cancer, metastatic castrate-sensitive prostate cancer) and has been shown to be a useful modulator of targeted protein ubiquitination and degradation via the ubiquitin-proteasome pathway.

Thus, in one aspect, this disclosure pertains to a pharmaceutical formulation comprising one or more pharmaceutically acceptable excipients and Compound A, or a pharmaceutically acceptable salt of Compound A, wherein the pharmaceutical formulation is in an oral dosage form that is selected from the group consisting of a tablet, a multiparticulate form (e.g. granules, pellets, or minitablets), and a capsule.

In another aspect, this disclosure pertains to a pharmaceutical formulation comprising one or more pharmaceutically acceptable excipients and Compound A, or a pharmaceutically acceptable salt of Compound A, wherein the pharmaceutical formulation is in an oral dosage form that is selected from the group consisting of a tablet, a sachet, and a capsule.

In still another aspect, this disclosure pertains to a pharmaceutical formulation comprising one or more pharmaceutically acceptable excipients and Compound A, wherein the pharmaceutical formulation is in an oral dosage form that is selected from the group consisting of a tablet, a multiparticulate form (e.g. granules, pellets, or minitablets), and a capsule.

In yet another aspect, this disclosure pertains to a pharmaceutical formulation comprising one or more pharmaceutically acceptable excipients and Compound A, wherein the pharmaceutical formulation is in an oral dosage form that is selected from the group consisting of a tablet, a sachet, and a capsule.

In another aspect, this disclosure pertains to a pharmaceutical formulation comprising one or more pharmaceutically acceptable excipients and a pharmaceutically acceptable salt of Compound A, wherein the pharmaceutical formulation is in an oral dosage form that is selected from the group consisting of a tablet, a multiparticulate form (e.g. granules, pellets, or minitablets), and a capsule.

In one aspect, this disclosure pertains to a pharmaceutical formulation comprising one or more pharmaceutically acceptable excipients and a pharmaceutically acceptable salt of Compound A, wherein the pharmaceutical formulation is in an oral dosage form that is selected from the group consisting of a tablet, a sachet, and a capsule.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and Compound A:

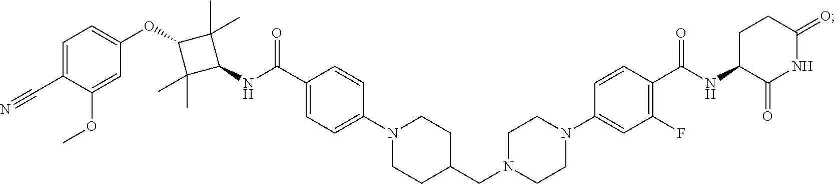 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   and wherein the extra-granular portion comprises:
a disintegrant;
a glidant; and
a lubricant.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

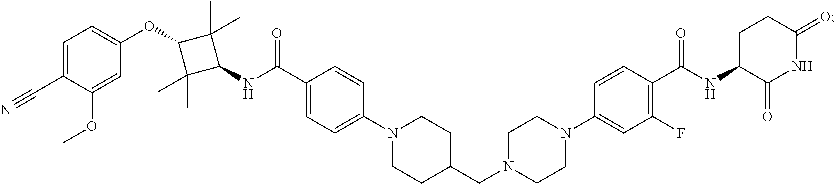 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   and wherein the extra-granular portion comprises:
a disintegrant;
a glidant; and
a lubricant.

In some embodiments, the oral dosage form provided herein is a tablet.

In one aspect, this disclosure pertains to a tablet comprising an intra-granular portion and an extra-granular portion, wherein the intra-granular portion comprises:
about 60.000% w/w to about 70.000% w/w of a solid dispersion comprising a release modifier and Compound A:

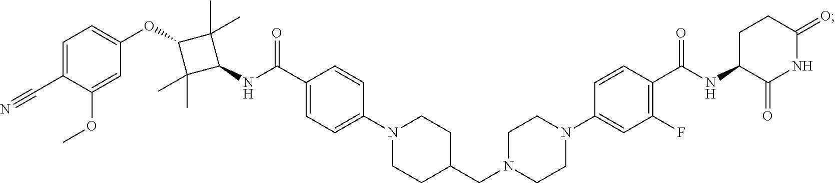 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   and wherein the extra-granular portion comprises:
a disintegrant;
a glidant; and
a lubricant.

In one aspect, this disclosure pertains to a tablet comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

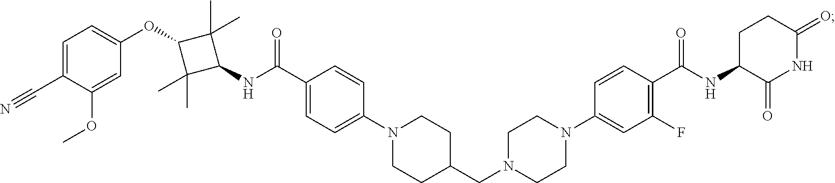 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant.
   and wherein the extra-granular portion comprises:
a disintegrant;
a glidant; and
a lubricant.

In one aspect, this disclosure pertains to a tablet comprising an intra-granular portion and an extra-granular portion, wherein the intra-granular portion comprises:
about 60.000% w/w to about 70.000% w/w of a solid dispersion comprising a release modifier and Compound A:

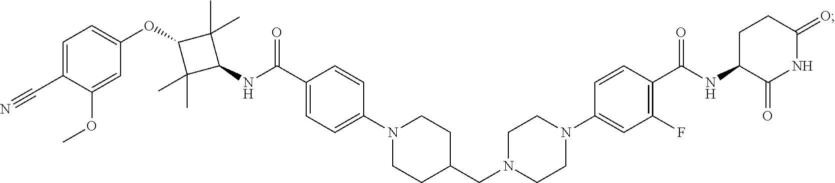 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a polysaccharide;
a sugar alcohol;
a disintegrant;
a glidant; and
a lubricant.
   and wherein the extra-granular portion comprises:
a disintegrant;
a glidant; and
a lubricant.

In one aspect, this disclosure pertains to a tablet comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

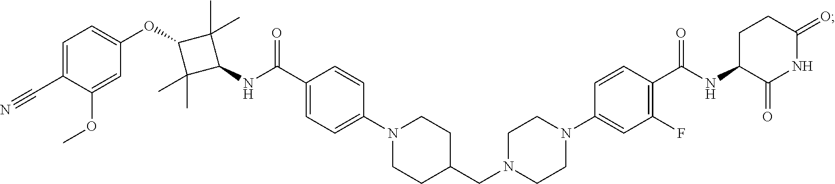 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a polysaccharide;
a sugar alcohol;
a disintegrant;
a glidant; and
a lubricant;
   and wherein the extra-granular portion comprises:
a disintegrant;
a glidant; and
a lubricant.

In one aspect, this disclosure pertains to a tablet comprising an intra-granular portion and an extra-granular portion, wherein the intra-granular portion comprises:
about 60.000% w/w to about 70.000% w/w of a solid dispersion comprising a release modifier and Compound A:

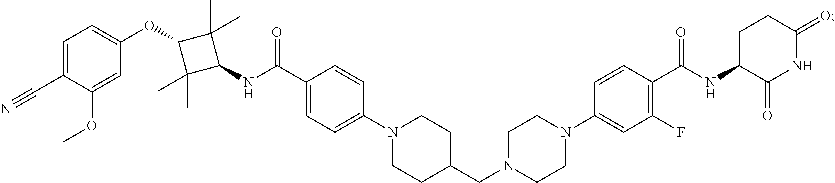 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

microcrystalline cellulose;
mannitol;
croscarmellose sodium;
crospovidone;
silicon dioxide;
colloidal silicon dioxide; and
sodium stearyl fumarate;
   and wherein the extra-granular portion comprises:
croscarmellose sodium;
colloidal silicon dioxide; and
sodium stearyl fumarate.

In one aspect, this disclosure pertains to a tablet comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

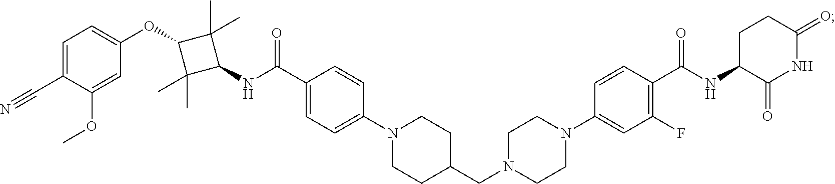 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

microcrystalline cellulose;
mannitol;
croscarmellose sodium;
crospovidone;
silicon dioxide;
colloidal silicon dioxide; and
sodium stearyl fumarate;
   and wherein the extra-granular portion comprises:
croscarmellose sodium;
colloidal silicon dioxide; and
sodium stearyl fumarate.

In one aspect, this disclosure pertains to a tablet comprising an intra-granular portion and an extra-granular portion, wherein the intra-granular portion comprises:
65.000% w/w to 68.000% w/w of a solid dispersion comprising a release modifier and Compound A:

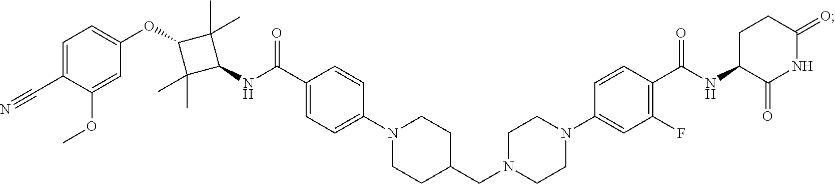 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

17.660% w/w to 21.660% w/w of one or more fillers;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In one aspect, this disclosure pertains to a tablet comprising an intra-granular portion and an extra-granular portion, wherein the intra-granular portion comprises:
65.000% w/w to 68.000% w/w of a solid dispersion comprising a release modifier and a pharmaceutically acceptable salt of Compound A:

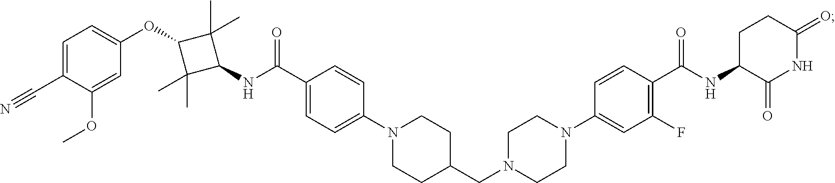 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

17.660% w/w to 21.660% w/w of one or more fillers;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In one aspect, this disclosure pertains to a tablet comprising an intra-granular portion and an extra-granular portion, wherein the intra-granular portion comprises:
65.000% w/w to 68.000% w/w of a solid dispersion comprising a release modifier and Compound A:

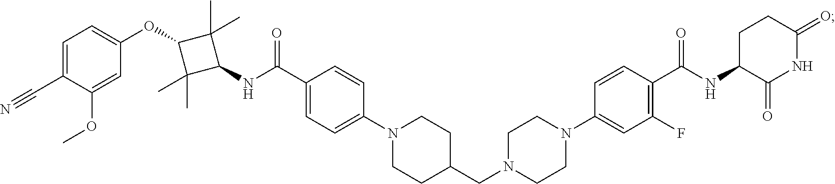 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w of a polysaccharide;
7.830% w/w to 11.830% w/w of a sugar alcohol;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In one aspect, this disclosure pertains to a tablet comprising an intra-granular portion and an extra-granular portion, wherein the intra-granular portion comprises:
65.000% w/w to 68.000% w/w of a solid dispersion comprising a release modifier and a pharmaceutically acceptable salt of Compound A:

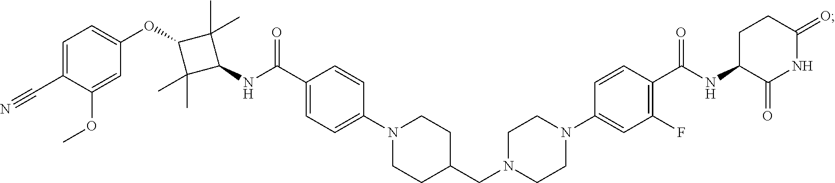 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w of a polysaccharide;
7.830% w/w to 11.830% w/w of a sugar alcohol;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In one aspect, this disclosure pertains to a tablet comprising an intra-granular portion and an extra-granular portion, wherein the intra-granular portion comprises:
65.000% w/w to 68.000% w/w of a solid dispersion comprising a release modifier and Compound A:

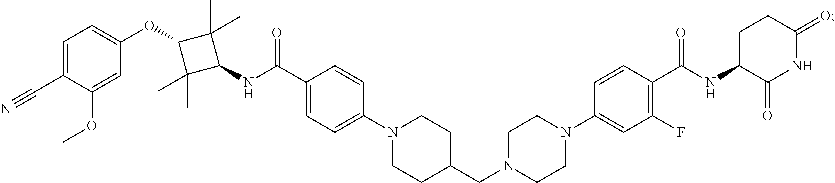 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w microcrystalline cellulose;
7.830% w/w to 11.830% w/w mannitol;
2.000% w/w to 6.000% w/w croscarmellose sodium;
2.000% w/w to 6.000% w/w crospovidone;
0.573% w/w to 0.773% w/w silicon dioxide;
0.250% w/w to 0.750% w/w colloidal silicon dioxide; and
0.100% w/w to 2.000% w/w sodium stearyl fumarate;
   and wherein the extra-granular portion comprises:
1.000% w/w to 4.000% w/w croscarmellose sodium;
0.100% w/w to 1.000% w/w colloidal silicon dioxide; and
0.100% w/w to 1.000% w/w sodium stearyl fumarate.

In one aspect, this disclosure pertains to a tablet comprising an intra-granular portion and an extra-granular portion, wherein the intra-granular portion comprises:
65.000% w/w to 68.000% w/w of a solid dispersion comprising a release modifier and a pharmaceutically acceptable salt of Compound A:

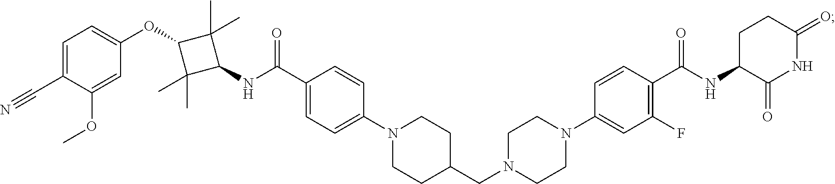 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w microcrystalline cellulose;
7.830% w/w to 11.830% w/w mannitol;
2.000% w/w to 6.000% w/w croscarmellose sodium;
2.000% w/w to 6.000% w/w crospovidone;
0.573% w/w to 0.773% w/w silicon dioxide;
0.250% w/w to 0.750% w/w colloidal silicon dioxide; and
0.100% w/w to 2.000% w/w sodium stearyl fumarate;
   and wherein the extra-granular portion comprises:
1.000% w/w to 4.000% w/w croscarmellose sodium;
0.100% w/w to 1.000% w/w colloidal silicon dioxide; and
0.100% w/w to 1.000% w/w sodium stearyl fumarate.

In one aspect, this disclosure pertains to a tablet comprising an intra-granular portion and an extra-granular portion, wherein the intra-granular portion comprises:
about 66.667% w/w of a solid dispersion comprising a release modifier and Compound A:

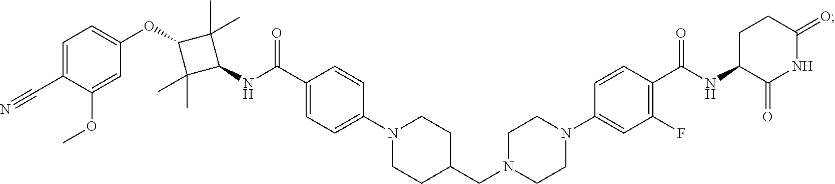 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In one aspect, this disclosure pertains to a tablet comprising an intra-granular portion and an extra-granular portion, wherein the intra-granular portion comprises:
about 66.667% w/w of a solid dispersion comprising a release modifier and a pharmaceutically acceptable salt of Compound A:

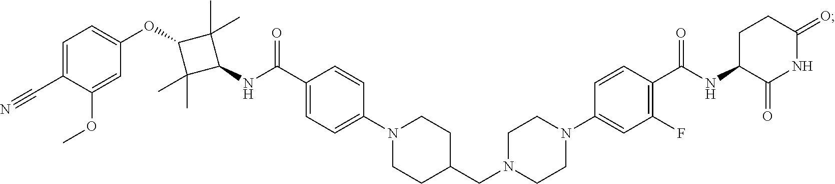 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In one aspect, this disclosure pertains to a tablet comprising an intra-granular portion and an extra-granular portion, wherein the intra-granular portion comprises:
about 66.667% w/w of a solid dispersion comprising a release modifier that is HPMCAS-M and Compound A:

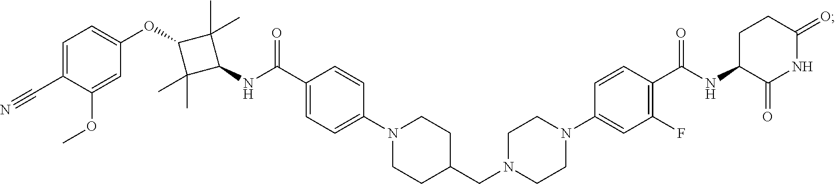 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In one aspect, this disclosure pertains to a tablet comprising an intra-granular portion and an extra-granular portion, wherein the intra-granular portion comprises:
about 66.667% w/w of a solid dispersion comprising a release modifier that is HPMCAS-M and a pharmaceutically acceptable salt of Compound A:

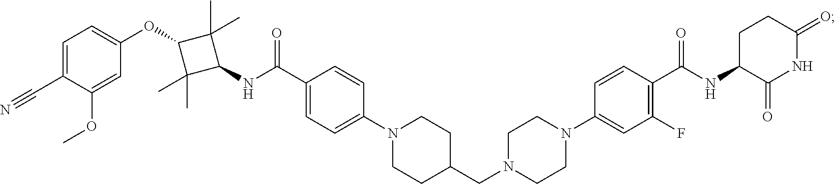 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In one aspect, this disclosure pertains to a method of treating prostate cancer in a subject in need thereof, wherein the method comprises administering a therapeutically effective amount of the dosage form or tablet of the disclosure to the subject.

In one aspect, this disclosure pertains to a method of manufacturing a tablet of the disclosure, wherein the method comprises:
(a) blending the intragranular ingredients to create a pre-blend;
(b) granulating the pre-blend to provide a milled blend;
(c) blending the milled blend with the extragranular ingredients to provide a final blend;
(d) compressing the final blend to provide the tablet or oral dosage form; and
(e) optionally, coating the tablet or oral dosage form. In one aspect, this disclosure pertains to a combination comprising:
an amorphous form of Compound A:

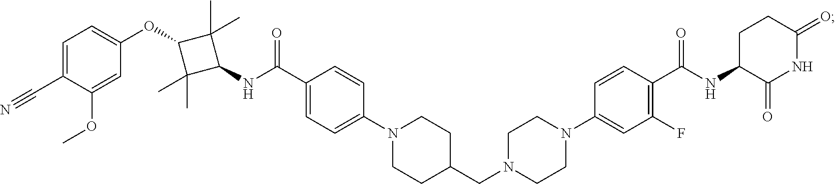 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
a release modifier.

In one aspect, this disclosure pertains to a combination comprising:
an amorphous form of a pharmaceutically acceptable salt of Compound A:

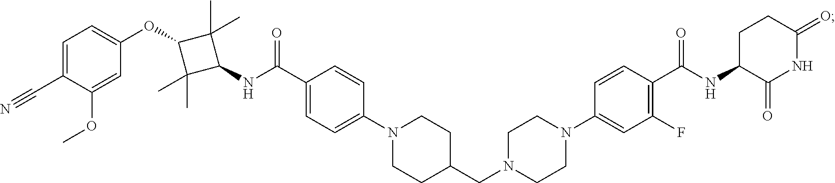 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
a release modifier.

In one aspect, this disclosure pertains to a method of manufacturing a combination of the disclosure, wherein the method comprises:
(a) dissolving Compound A, or a pharmaceutically acceptable salt of Compound A, and the release modifier in a solvent to provide a solution;
(b) spray-drying the solution to provide a wet spray-dried dispersion; and
(c) drying the wet spray-dried dispersion to provide the combination.

### BRIEF DESCRIPTION OF THE DRAWINGS

The accompanying drawings, which are incorporated into and form a part of the specification, illustrates several embodiments of the present disclosure and, together with the description, serves to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figure showing illustrative embodiments of the disclosure, in which:

FIG. 1, FIG. 2, and FIG. 3 are flow charts that show exemplary schemes of preparing tablets of the disclosure.

### DETAILED DESCRIPTION

Provided herein is an oral dosage form comprising an intra-granular portion and an extra-granular portion, wherein the intra-granular portion comprises: a solid dispersion comprising a release modifier and Compound A; a filler; a disintegrant; a glidant; and a lubricant, and wherein the extra-granular portion comprises: a disintegrant; a glidant; and a lubricant.

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety for all purposes.

### Definitions

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the present disclosure. The upper and lower limits of these smaller ranges may independently be included in the smaller ranges subject to any specifically excluded limit in the stated range. Where the stated range includes one or both of the limits, ranges excluding either or both of those included limits are also included in the disclosure. When a range is expressed as being between two values, both endpoints are understood to be included in the range unless context dictates otherwise.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an oral dosage form” means one oral dosage form or more than one oral dosage form.

The phrase “and/or,” as used herein in the specification and in the claims, should be understood to mean “either or both”. Other elements may optionally be present other than the elements specifically identified by the “and/or” clause. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The term “about” is used herein to mean approximately, in the region of, roughly or around. When the term “about” is used in conjunction with a numerical range, it modifies that range by extending the boundaries above and below the numerical values set forth. In general, unless otherwise specified or unless the context clearly dictates otherwise, the term “about” is used herein to modify a numerical value above and below the stated value by a variance of 20%. In some embodiments, the term “about” refers to a variance of 10%, a variance of 5%, a variance of 3%, or a variance of 1%. For example, “about 35.00% w/w” is equivalent to a range of 30.00% w/w to 40.00% w/w (10% variance), or a range of 32.50% w/w to 37.50% w/w (5% variance), or a range of 34.50% w/w to 35.50% w/w (1% variance).

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

Prostate cancer is the uncontrolled growth of cancerous cells in the prostate gland. In some embodiments, the prostate cancer is metastatic prostate cancer, castrate-resistant prostate cancer, metastatic castrate-resistant prostate cancer, castrate-sensitive prostate cancer, metastatic castrate-sensitive prostate cancer, prostate cancer naïve to novel hormonal agents (NHA), castrate-sensitive prostate cancer naïve to novel hormonal agents (NHA), castrate-resistant prostate cancer naïve to novel hormonal agents (NHA), metastatic prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA).

Metastatic prostate cancer, or metastases, refers to prostate cancer that has spread beyond the prostate to other parts of the body, e.g., bones, lymph nodes, liver, lungs, brain.

Castrate-resistant prostate cancer or castration-resistant prostate cancer (or prostate cancer that is castrate- or castration-resistant) is a type of prostate cancer that keeps growing even when the amount of testosterone in the body is reduced to very low levels.

Metastatic castrate-resistant prostate cancer is a type of prostate cancer that has metastasized and continues to grow even when the amount of testosterone in the body is reduced to very low levels.

Castrate-sensitive prostate cancer or castration-sensitive prostate cancer (CSPC), or prostate cancer that is castrate- or castration-sensitive, is prostate cancer that can be controlled by reducing the amount of androgens (male hormones) in the body (e.g., through castration) and/or prostate cancer that requires androgens to grow and stops growing when androgens are not present. CSPC is also referred to as androgen-dependent prostate cancer, androgen-sensitive prostate cancer, or hormone-sensitive prostate cancer (HSPC).

Metastatic castrate-sensitive prostate cancer is a type of castrate-sensitive prostate cancer that has metastasized and requires androgens to grow or can be controlled by reducing the amount of androgens in the body (e.g., through castration).

Prostate cancer naïve to novel hormonal agents (NHA) is prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Metastatic prostate cancer naïve to novel hormonal agents (NHA) is metastatic prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Castrate-resistant prostate cancer naïve to novel hormonal agents (NHA) is castrate-resistant prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Castrate-sensitive prostate cancer naïve to novel hormonal agents (NHA) is castrate-sensitive prostate cancer that has not been previously treated with second-generation antiandrogens such as androgen biosynthesis inhibitors or androgen receptor blockers. In some embodiments, the androgen biosynthesis inhibitor is abiraterone or abiraterone acetate). In some embodiments, the androgen receptor blocker is enzalutamide, darolutamide, or apalutamide.

Metastatic castrate-resistant prostate cancer naïve to novel hormonal agents (NHA) is metastatic castrate-resistant prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Metastatic castrate-sensitive prostate cancer naïve to novel hormonal agents (NHA) is metastatic castrate-sensitive prostate cancer that has not been previously treated with second-generation antiandrogens such as androgen biosynthesis inhibitors or androgen receptor blockers. In some embodiments, the androgen biosynthesis inhibitor is abiraterone or abiraterone acetate). In some embodiments, the androgen receptor blocker is enzalutamide, darolutamide, or apalutamide.

The terms “crystalline form,” “morphic form,” and “polymorph” are each understood to mean any solid that has a short or long range order of the molecules, atoms or ions in a fixed lattice arrangement. Therefore, crystals may be in e.g., a triclinic, monoclinic, orthorhombic, tetragonal, rhombohedral, hexagonal or cubic crystal form or mixtures thereof. In some embodiments, Compound A is present in nanocrystalline form.

The term “amorphous” form refers to solids of disordered arrangements of molecules and that do not possess a distinguishable crystal lattice.

The terms “co-administration”, “co-administering”, and “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein are co-administered in combination with at least one additional therapeutic agent, especially including an additional anticancer agent. In particularly preferred aspects, the co-administration of compounds results in improved activity or synergistic activity and/or therapy, including anticancer activity.

The term “compound”, as used herein, unless otherwise indicated, refers to any specific chemical compound disclosed herein, including deuterated forms thereof where applicable, in context. Deuterated compounds contemplated are those in which one or more of the hydrogen atoms contained in the drug molecule have been replaced by deuterium. Unless the context indicates otherwise, the term compound refers to Compound A.

Compound A of the present disclosure refers to 4-(4-((1-(4-(((1R,3R)-3-(4-cyano-3-methoxyphenoxy)-2,2,4,4-tetramethylcyclobutyl)carbamoyl)phenyl)piperidin-4-yl)methyl)piperazin-1-yl)-N-((S)-2,6-dioxopiperidin-3-yl)-2-fluorobenzamide, which has the following structure:

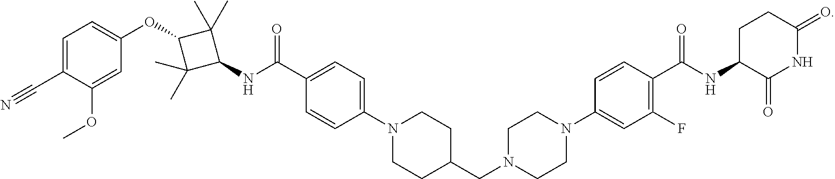 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Compound A can be prepared as described in U.S. Pat. No. 11,883,393 or U.S. patent application Ser. No. 19/072,307, both of which are incorporated herein by reference in their entireties.

“Pharmaceutically acceptable salt,” as used herein with respect to a compound of the disclosure, means a salt form of the compound of the disclosure as well as hydrates of the salt form with one or more water molecules present. Such salt and hydrated forms retain the biological activity of the compound of the disclosure and are not biologically or otherwise undesirable, i.e., exhibit minimal, if any, toxicological effects. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

In any of the oral dosage forms disclosed herein, Compound A may be present in the form of a co-crystal. “Co-crystal,” as used herein, means a crystalline material composed of two or more unique solids at room temperature, each of which has distinctive physical characteristics such as structure, melting point, and heats of fusion, hygroscopicity, solubility, and stability. A co-crystal can be produced according to a per se known co-crystallization method. The term co-crystal also refers to a multicomponent system in which there exists a host API (active pharmaceutical ingredient) molecule or molecules, such as a Compound A, and a guest (or co-former) molecule or molecules. In some embodiments said pharmaceutically acceptable co-crystal of Compound A with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

In any of the oral dosage forms disclosed herein, Compound A may be present in the form of a eutectic mixture. The term “eutectic mixture,” as used herein, means a homogeneous mixture of two or more components that react to form a single phase as a melt or solidify or form a liquid compound (single phase) at a single temperature that is lower than the melting point of any of the components.

The term “ubiquitin ligase” refers to a family of proteins that facilitate the transfer of ubiquitin to a specific substrate protein, targeting the substrate protein for degradation. For example, cereblon is an E3 ubiquitin ligase protein that alone or in combination with an E2 ubiquitin-conjugating enzyme causes the attachment of ubiquitin to a lysine on a target protein. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Such polyubiquitination marks the target proteins for degradation by the proteasome.

“Comprising” or “comprises” as applied to a particular dosage form, composition, use, method or process described or claimed herein means that the dosage form, composition, use, method, or process includes all of the recited elements in a specific description or claim, but does not exclude other elements. “Consists essentially of” and “consisting essentially of” means that the described or claimed composition, dosage form, method, use, or process does not exclude other materials or steps that do not materially affect basic novel characteristics of the claimed composition, dosage form, method, use, or process, such as the recited physical, pharmacological, pharmacokinetic properties or therapeutic effects of the composition, dosage form, method, use, or process. “Consists of” and “consisting of” means the exclusion of more than trace elements of other ingredients and substantial method or process steps.

All percentages provided herein are percentages by weight and may be abbreviated “wt %” or “% w/w”, unless indicated otherwise. It is understood that, when a composition is described in which ranges are provided for multiple components, the total amount of all recited components does not exceed 100%. It is within the ability of a skilled artisan to adjust the amounts within the ranges provided to accomplish this.

Unless stated otherwise, all weight percentages related to dosage forms and tablets of the disclosure provided herein are referencing the total weight of the uncoated dosage forms or uncoated tablets.

### Oral Dosage Forms

In one aspect, this disclosure provides an oral dosage form of Compound A. In some embodiments, the oral dosage form is selected from the group consisting of a tablet, a multiparticulate form (e.g. granules, pellets, or minitablets), or a capsule. In some embodiments, the oral dosage form is selected from the group consisting of a tablet, a sachet, and a capsule. In some embodiments, the oral dosage form is a tablet.

“Oral dosage form” as used herein refers to a pharmaceutical drug product that contains a specified amount (dose) of Compound A as the active ingredient, or a pharmaceutically acceptable salt thereof, and inactive components (excipients), formulated into a particular configuration that is suitable for oral administration, such as an oral tablet, liquid, or capsule. In some embodiments, the oral dosage form comprises a tablet. In some embodiments, the oral dosage form comprises a tablet. In some embodiments, the oral dosage form comprises a film-coated tablet. In some embodiments, the oral dosage form comprises a tablet that can be scored. In some embodiments, the oral dosage form comprises a sublingual tablet. In some embodiments, the oral dosage form comprises a capsule, which can be taken intact or used as a sprinkle onto food (e.g., applesauce or yogurt). In some embodiments, the oral dosage form comprises a multiparticulate form (e.g. granules, pellets, or minitablets). In some embodiments, the oral dosage form comprises a sachet.

“Oral administration” as used herein refers to enteral, buccal, sublabial, or sublingual medications in the form of tablets, capsules, syrups, powders, granules, pastilles, solutions, tinctures, elixirs, emulsions, hydrogels, teas, films, disintegrating tablets, mouthwashes, and others. In some embodiments, oral administration refers to enteral, buccal, sublabial, or sublingual medications in the form of softgels and suspensions. In some embodiments, oral administration refers to enteral, buccal, sublabial, or sublingual medications in the form of lipid-based formulations, comprising, for example, lipids, cosolvents and surfactants.

Suitable forms for oral administration may include one or more pharmaceutically acceptable excipients, including, for example, carriers, fillers, surfactants, diluents, sweeteners, disintegrants, binders, lubricants, glidants, colorants, flavors, stabilizing agents, coatings, or any mixtures thereof.

In one aspect, this disclosure pertains to an oral dosage form comprising:
Compound A;
a release modifier; and
one or more components selected from the group consisting of a filler, a disintegrant, a glidant, and a lubricant.

In one aspect, this disclosure pertains to an oral dosage form comprising:
Compound A;
a release modifier;
a filler;
a disintegrant;
a glidant; and
a lubricant.

In one aspect, this disclosure pertains to an oral dosage form comprising an intra-granular portion and an extra-granular portion, wherein the intra-granular portion comprises:
a solid dispersion comprising a release modifier and Compound A;
a filler;
a disintegrant;
a glidant; and
a lubricant;
   and wherein the extra-granular portion comprises:
a disintegrant;
a glidant; and
a lubricant.

In one aspect, this disclosure pertains to an oral dosage form comprising an intra-granular portion and an extra-granular portion, wherein the intra-granular portion comprises:
about 20.000% w/w to about 80.000% w/w, optionally about 60.000% w/w to about 70.000% w/w, of a solid dispersion comprising a release modifier and Compound A:

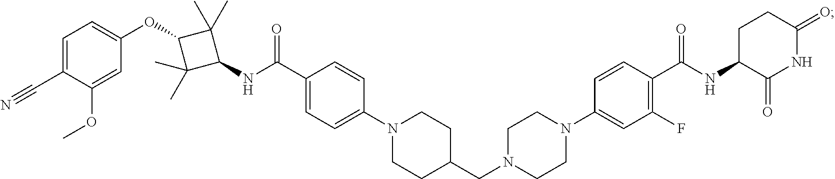 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   and wherein the extra-granular portion comprises:
a disintegrant;
a glidant; and
a lubricant.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and Compound A:

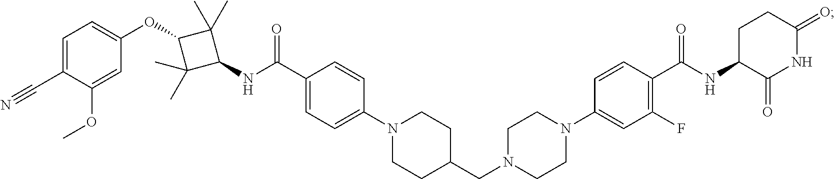 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   and wherein the extra-granular portion comprises:
a disintegrant;
a glidant; and
a lubricant.

In some embodiments, the oral dosage form (e.g., tablet) further comprises a film coat. In some embodiments, the film coat comprises at least one of the following: a film forming agent, an opacifier, a colorant, an anti-sticking agent, a plasticizer, and a wetting agent.

Thus, in one aspect, this disclosure pertains to a tablet comprising an intra-granular portion and an extra-granular portion, wherein the intra-granular portion comprises:
about 60.000% w/w to about 70.000% w/w of a solid dispersion comprising a release modifier and Compound A:

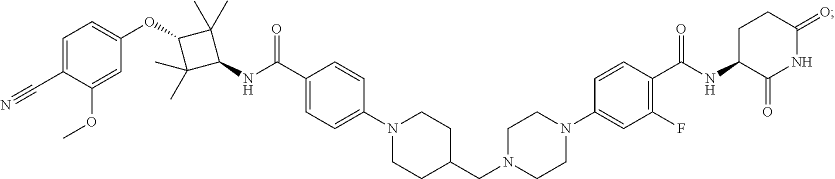 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   and wherein the extra-granular portion comprises:
a disintegrant;
a glidant; and
a lubricant,
wherein the tablet further comprises a film coat.

In one aspect, this disclosure pertains to an oral dosage form comprising an intra-granular portion and an extra-granular portion, wherein the intra-granular portion comprises:
62.000% w/w to 68.000% w/w of a solid dispersion comprising a release modifier and Compound A:

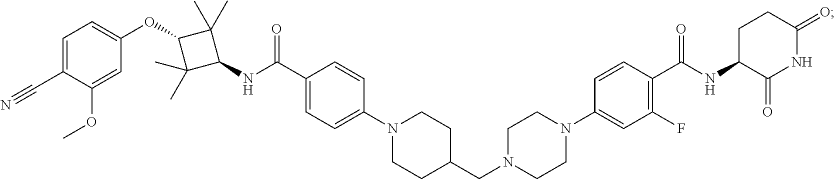 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.660% w/w microcrystalline cellulose;
2.830% w/w to about 11.830% w/w mannitol;
2.000% w/w to 10.000% w/w croscarmellose sodium;
1.000% w/w to about 7.000% w/w crospovidone;
0.250% w/w to 1.500% w/w colloidal silicon dioxide; and
0.100% w/w to 2.000% w/w sodium stearyl fumarate;
   wherein the extra-granular portion comprises:
1.000% w/w to 4.000% w/w croscarmellose sodium;
0.100% w/w to 1.000% w/w colloidal silicon dioxide; and
0.100% w/w to 1.000% w/w sodium stearyl fumarate;
   and wherein the film coat is 1.000% w/w to 6.000% w/w of the tablet.

In some embodiments, the film is a polyvinyl alcohol-based film-coat with titanium dioxide, talcum, macrogol, and ferric oxides.

In one aspect, this disclosure pertains to a tablet comprising an intra-granular portion, an extra-granular portion, and a film coat, wherein the intra-granular portion comprises:
about 60.000% w/w to about 70.000% w/w of a solid dispersion comprising a release modifier and Compound A:

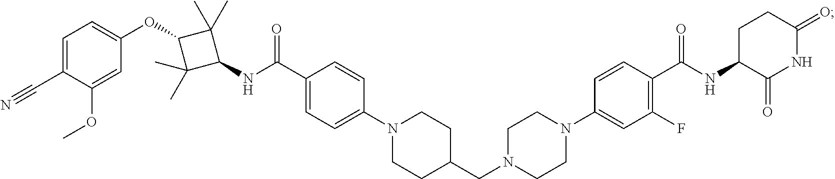 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   wherein the extra-granular portion comprises:
a disintegrant;
a glidant; and
a lubricant,
   and wherein the film coat comprises:
a film forming agent,
an opacifier,
a colorant,
an anti-sticking agent,
a plasticizer,
and a wetting agent.

In some embodiments, the film coat comprises:
a film forming agent selected from polyvinyl alcohol, cellulose derivatives (e.g., hydroxypropyl methylcellulose, hydroxyethyl methylcellulose, and hydroxyethyl cellulose), polyvinylpyrrolidone (PVP), and a copolymer of methacrylic acid and acrylic acid (e.g., poly(methacrylic acid, methyl methacrylate) 1:1; poly(methacrylic acid, methyl methacrylate) 1:2; and poly(butyl methacrylate, (2-dimethylaminoethyl) methacrylate, methyl methacrylate 1:2:1),
an opacifier selected from titanium dioxide, calcium carbonate, and dicalcium phosphate,
a colorant selected from iron oxide (ferric oxide), FD&C Blue No. 1 Aluminum Lake, FD&C Blue No. 2 (indigotine), and titanium dioxide,
an anti-sticking agent selected from talcum, silicon dioxide, colloidal silicon dioxide, magnesium silicate, and magnesium trisilicate,
a plasticizer selected from macrogol (PEG) polyvinyl alcohol graft copolymer, polyethylene glycol, and glycerol esters of fatty acids (e.g., glyceryl monocaprylocaprate),
and a wetting agent selected from glyceryl mono and dicprylocaprate, sodium lauryl sulfate, and lecithin.

In some embodiments, the film coat is about 1-6% w/w of the tablet.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

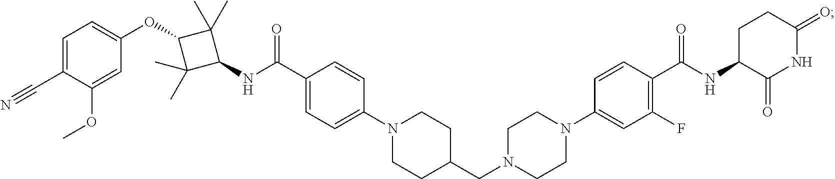 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a filler;
a disintegrant;
a glidant; and
a lubricant;
   and wherein the extra-granular portion comprises:
a disintegrant;
a glidant; and
a lubricant.

In some embodiments, the intra-granular portion comprises a first filler and a second filler.

In some embodiments, the intra-granular portion comprises a single filler.

In some embodiments, the first filler is microcrystalline cellulose.

In some embodiments, the second filler is mannitol.

In some embodiments, the intra-granular portion comprises a single filler, which is microcrystalline cellulose.

In some embodiments, the intra-granular portion comprises a first disintegrant and a second disintegrant.

In some embodiments, the intra-granular portion comprises a single disintegrant.

In some embodiments, the first disintegrant in the intra-granular portion is a carboxymethylcellulose derivative.

In some embodiments, the first disintegrant in the intra-granular portion is croscarmellose sodium.

In some embodiments, the second disintegrant in the intra-granular portion is crospovidone.

In some embodiments, the intra-granular portion comprises a single disintegrant, which is croscarmellose sodium.

In some embodiments, the disintegrant in the extra-granular portion is a carboxymethylcellulose derivative.

In some embodiments, the disintegrant in the extra-granular portion is croscarmellose sodium.

In some embodiments, the intra-granular portion comprises a first glidant and/or a second glidant.

In some embodiments, the intra-granular portion comprises a single glidant that is colloidal silicon dioxide.

In some embodiments, the intra-granular portion comprises a first glidant and a second glidant.

In some embodiments, the first glidant and/or the second glidant in the intra-granular portion is selected from the group consisting of silicon dioxide, colloidal silicon dioxide, and sodium stearyl fumarate.

In some embodiments, first glidant in the intra-granular portion is silicon dioxide.

In some embodiments, the second glidant in the intra-granular portion is colloidal silicon dioxide.

In some embodiments, the glidant in the extra-granular portion is colloidal silicon dioxide.

In some embodiments, the extra-granular portion comprises a first glidant and a second glidant.

In some embodiments, the first glidant in the extra-granular portion is silicon dioxide.

In some embodiments, the second glidant in the extra-granular portion is colloidal silicon dioxide.

In some embodiments, the lubricant in the intra-granular portion is sodium stearyl fumarate.

In some embodiments, the lubricant in the extra-granular portion is sodium stearyl fumarate.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and Compound A:

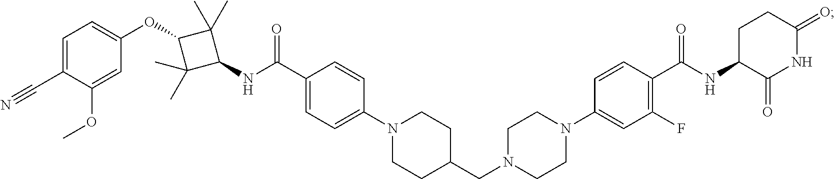 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a polysaccharide;
a sugar alcohol;
a disintegrant;
a glidant; and
a lubricant;
   and wherein the extra-granular portion comprises:
a disintegrant;
a glidant; and
a lubricant.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

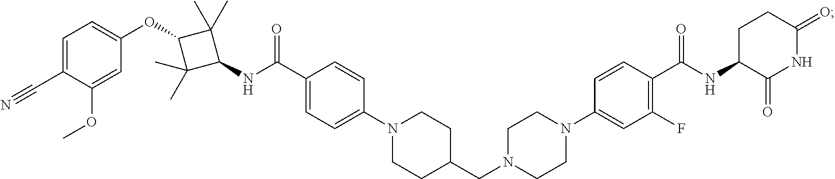 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a polysaccharide;
a sugar alcohol;
a disintegrant;
a glidant; and
a lubricant;
   and wherein the extra-granular portion comprises:
a disintegrant;
a glidant; and
a lubricant.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

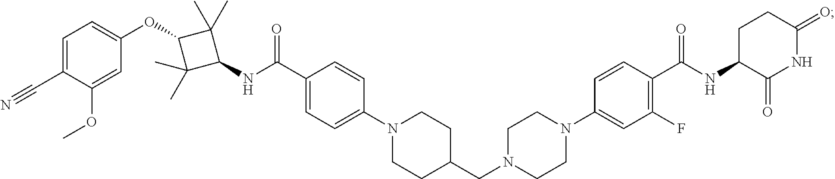 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

a polysaccharide;
a sugar alcohol;
a disintegrant;
a glidant; and
a lubricant;
   and wherein the extra-granular portion comprises:
a polysaccharide;
a disintegrant;
a glidant; and
a lubricant.

In some embodiments, the polysaccharide in both the intra-granular portion and the extra-granular portion is microcrystalline cellulose.

In some embodiments, the polysaccharide is microcrystalline cellulose.

In some embodiments, the sugar alcohol is mannitol.

In some embodiments, the intra-granular portion comprises a first disintegrant and a second disintegrant.

In some embodiments, the first disintegrant in the intra-granular portion is a carboxymethylcellulose derivative.

In some embodiments, the first disintegrant in the intra-granular portion is croscarmellose sodium.

In some embodiments, the second disintegrant in the intra-granular portion is crospovidone.

In some embodiments, the disintegrant in the extra-granular portion is a carboxymethylcellulose derivative.

In some embodiments, the disintegrant in the extra-granular portion is croscarmellose sodium.

In some embodiments, the intra-granular portion comprises a first glidant and/or a second glidant.

In some embodiments, the intra-granular portion comprises a single glidant that is colloidal silicon dioxide.

In some embodiments, the intra-granular portion comprises a first glidant and a second glidant.

In some embodiments, the first glidant and/or the second glidant in the intra-granular portion is selected from the group consisting of silicon dioxide, colloidal silicon dioxide, and sodium stearyl fumarate.

In some embodiments, first glidant in the intra-granular portion is silicon dioxide.

In some embodiments, the second glidant in the intra-granular portion is colloidal silicon dioxide.

In some embodiments, the glidant in the extra-granular portion is colloidal silicon dioxide.

In some embodiments, the extra-granular portion comprises a first glidant and a second glidant.

In some embodiments, the first glidant in the extra-granular portion is silicon dioxide.

In some embodiments, the second glidant in the extra-granular portion is colloidal silicon dioxide.

In some embodiments, the lubricant in the intra-granular portion is sodium stearyl fumarate.

In some embodiments, the lubricant in the extra-granular portion is sodium stearyl fumarate.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and Compound A:

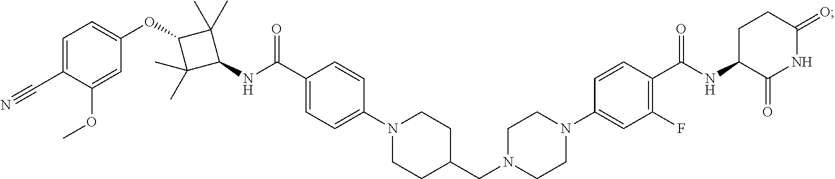 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

microcrystalline cellulose;
mannitol;
croscarmellose sodium;
crospovidone;
silicon dioxide;
colloidal silicon dioxide; and
sodium stearyl fumarate;
   and wherein the extra-granular portion comprises:
croscarmellose sodium;
colloidal silicon dioxide; and
sodium stearyl fumarate.

In one aspect, this disclosure pertains to an oral dosage form comprising an intra-granular portion and an extra-granular portion, wherein the intra-granular portion comprises:
about 60.000% w/w to about 70.000% w/w of a solid dispersion comprising a release modifier and a pharmaceutically acceptable salt of Compound A:

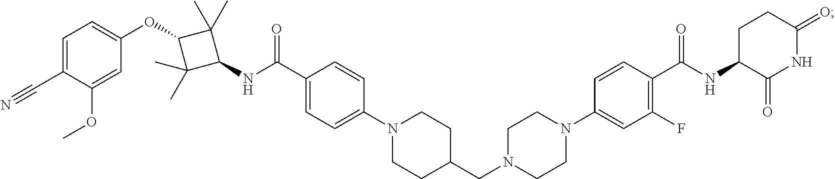 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

microcrystalline cellulose;
mannitol;
croscarmellose sodium;
crospovidone;
silicon dioxide;
colloidal silicon dioxide; and
sodium stearyl fumarate;
   and wherein the extra-granular portion comprises:
croscarmellose sodium;
colloidal silicon dioxide; and
sodium stearyl fumarate.

In some embodiments, the amount of microcrystalline cellulose in the intra-granular portion of the oral dosage form (e.g., tablet) is about 4.830% w/w to about 33.275% w/w, e.g., 9.830% w/w or 10.995% w/w.

In some embodiments, the amount of microcrystalline cellulose in the intra-granular portion of the oral dosage form is about 4.830% w/w to about 14.830% w/w, e.g., 9.830% w/w.

In some embodiments, the amount of mannitol in the intra-granular portion of the oral dosage form is about 2.830% w/w to about 14.830% w/w, e.g., 3.665% w/w or 9.830% w/w.

In some embodiments, the amount of mannitol in the intra-granular portion of the oral dosage form is about 4.830% w/w to about 14.830% w/w, e.g., 9.830% w/w.

In some embodiments, the amount of croscarmellose sodium in the intra-granular portion of the oral dosage form is about 1.000% w/w to about 7.000% w/w, e.g., 4.000% w/w.

In some embodiments, the amount of crospovidone in the intra-granular portion of the oral dosage form is about 1.000% w/w to about 7.000% w/w, e.g., 4.000% w/w.

In some embodiments, the amount of croscarmellose sodium in the extra-granular portion of the oral dosage form is about 1.500% w/w to about 5.500% w/w, e.g., 2.500% w/w.

In some embodiments, the amount of silicon dioxide in the intra-granular portion of the oral dosage form is about 0.573% w/w to about 0.773% w/w, e.g., 0.673% w/w.

In some embodiments, the amount of colloidal silicon dioxide in the intra-granular portion of the oral dosage form is about 0.250% w/w to about 0.750% w/w, e.g., 0.500% w/w.

In some embodiments, the amount of microcrystalline cellulose in the extra-granular portion of the oral dosage form (e.g., tablet) is about 2.000% w/w to about 6.000% w/w, e.g., 5.000% w/w.

In some embodiments, the amount of colloidal silicon dioxide in the extra-granular portion of the oral dosage form is about 0.250% w/w to about 0.750% w/w, e.g., 0.500% w/w.

In some embodiments, the amount of sodium stearyl fumarate in the intra-granular portion of the oral dosage form is about 0.500% w/w to about 1.500% w/w, e.g., 1.000% w/w.

In some embodiments, the amount of sodium stearyl fumarate in the extra-granular portion of the oral dosage form is about 0.250% w/w to about 0.750% w/w, e.g., 0.500% w/w.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and Compound A:

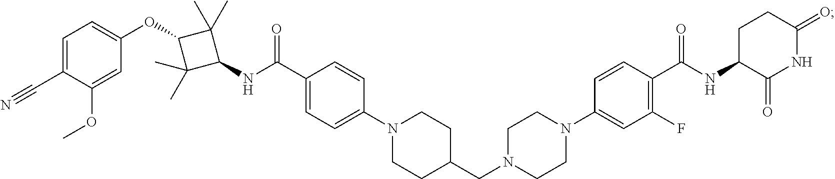 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

17.660% w/w to 21.660% w/w of one or more fillers;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and a pharmaceutically acceptable salt of Compound A:

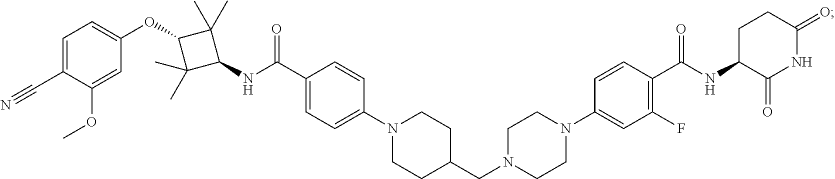 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

17.660% w/w to 21.660% w/w of one or more fillers;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In some embodiments, the intra-granular portion comprises two fillers.

In some embodiments, the first filler is microcrystalline cellulose.

In some embodiments, the second filler is mannitol.

In some embodiments, the intra-granular portion comprises a first disintegrant and a second disintegrant.

In some embodiments, the first disintegrant in the intra-granular portion is a carboxymethylcellulose derivative.

In some embodiments, the first disintegrant in the intra-granular portion is croscarmellose sodium.

In some embodiments, the second disintegrant in the intra-granular portion is crospovidone.

In some embodiments, the disintegrant in the extra-granular portion is a carboxymethylcellulose derivative.

In some embodiments, the disintegrant in the extra-granular portion is croscarmellose sodium.

In some embodiments, the intra-granular portion comprises a first glidant and/or a second glidant.

In some embodiments, the intra-granular portion comprises a first glidant and a second glidant.

In some embodiments, the first glidant and/or the second glidant in the intra-granular portion is selected from the group consisting of silicon dioxide, colloidal silicon dioxide, and sodium stearyl fumarate.

In some embodiments, first glidant in the intra-granular portion is silicon dioxide.

In some embodiments, the second glidant in the intra-granular portion is colloidal silicon dioxide.

In some embodiments, the glidant in the extra-granular portion is colloidal silicon dioxide.

In some embodiments, the extra-granular portion comprises a first glidant and a second glidant.

In some embodiments, the first glidant in the extra-granular portion is silicon dioxide.

In some embodiments, the second glidant in the extra-granular portion is colloidal silicon dioxide.

In some embodiments, the lubricant in the intra-granular portion is sodium stearyl fumarate.

In some embodiments, the lubricant in the extra-granular portion is sodium stearyl fumarate.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and Compound A:

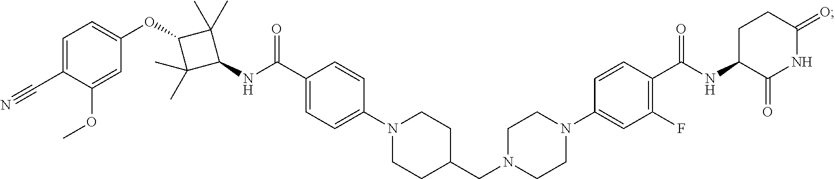 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w of a polysaccharide;
7.830% w/w to 11.830% w/w of a sugar alcohol;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and a pharmaceutically acceptable salt of Compound A:

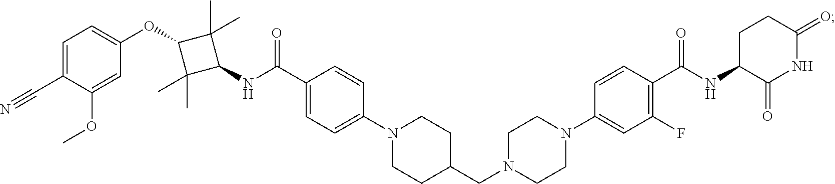 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w of a polysaccharide;
7.830% w/w to 11.830% w/w of a sugar alcohol;
6.000% w/w to 10.000% w/w of one or more disintegrants;
0.173% w/w to 2.173% w/w of one or more glidants; and
0.100% w/w to 2.000% w/w of a lubricant;
   and wherein the extra-granular portion comprises:
1.000% w/w to 4.000% w/w of a disintegrant;
0.100% w/w to 1.000% w/w of a glidant; and
0.100% w/w to 1.000% w/w of a lubricant.

In some embodiments, the polysaccharide is microcrystalline cellulose.

In some embodiments, the sugar alcohol is mannitol.

In some embodiments, the intra-granular portion comprises a first disintegrant and a second disintegrant.

In some embodiments, the first disintegrant in the intra-granular portion is a carboxymethylcellulose derivative.

In some embodiments, the first disintegrant in the intra-granular portion is croscarmellose sodium.

In some embodiments, the second disintegrant in the intra-granular portion is crospovidone.

In some embodiments, the disintegrant in the extra-granular portion is a carboxymethylcellulose derivative.

In some embodiments, the disintegrant in the extra-granular portion is croscarmellose sodium.

In some embodiments, the intra-granular portion comprises a first glidant and/or a second glidant.

In some embodiments, the intra-granular portion comprises a first glidant and a second glidant.

In some embodiments, the first glidant and/or the second glidant in the intra-granular portion is selected from the group consisting of silicon dioxide, colloidal silicon dioxide, and sodium stearyl fumarate.

In some embodiments, first glidant in the intra-granular portion is silicon dioxide.

In some embodiments, the second glidant in the intra-granular portion is colloidal silicon dioxide.

In some embodiments, the glidant in the extra-granular portion is colloidal silicon dioxide.

In some embodiments, the extra-granular portion comprises a first glidant and a second glidant.

In some embodiments, the first glidant in the extra-granular portion is silicon dioxide.

In some embodiments, the second glidant in the extra-granular portion is colloidal silicon dioxide.

In some embodiments, the lubricant in the intra-granular portion is sodium stearyl fumarate.

In some embodiments, the lubricant in the extra-granular portion is sodium stearyl fumarate.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and Compound A:

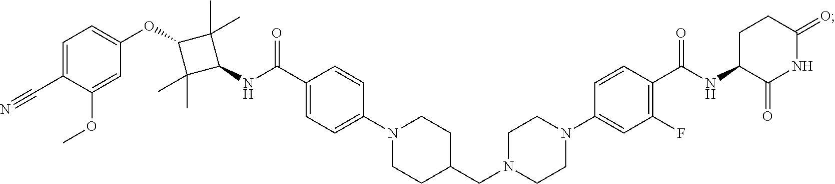 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 23.660% w/w microcrystalline cellulose;
2.830% w/w to about 14.830% w/w mannitol;
2.000% w/w to 12.000% w/w croscarmellose sodium;
1.000% w/w to about 7.000% w/w crospovidone;
0.573% w/w to 0.773% w/w silicon dioxide;
0.250% w/w to 0.750% w/w colloidal silicon dioxide; and
0.100% w/w to 2.000% w/w sodium stearyl fumarate;
   and wherein the extra-granular portion comprises:
3.830% w/w to 23.660% w/w microcrystalline cellulose;
1.000% w/w to 4.000% w/w croscarmellose sodium;
0.100% w/w to 1.000% w/w colloidal silicon dioxide; and
0.100% w/w to 1.000% w/w sodium stearyl fumarate.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and Compound A:

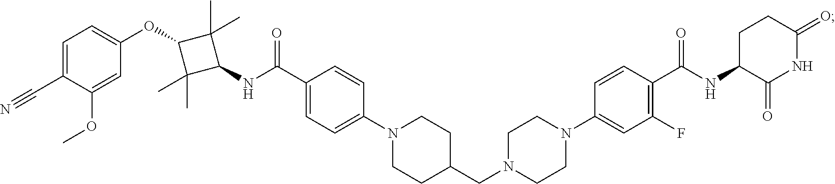 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 23.660% w/w microcrystalline cellulose;
2.000% w/w to 12.000% w/w croscarmellose sodium;
0.573% w/w to 0.773% w/w silicon dioxide;
0.250% w/w to 0.750% w/w colloidal silicon dioxide; and
0.100% w/w to 2.000% w/w sodium stearyl fumarate;
   and wherein the extra-granular portion comprises:
7.830% w/w to 23.660% w/w microcrystalline cellulose;
1.000% w/w to 4.000% w/w croscarmellose sodium;
0.100% w/w to 1.000% w/w colloidal silicon dioxide; and
0.100% w/w to 1.000% w/w sodium stearyl fumarate.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and Compound A:

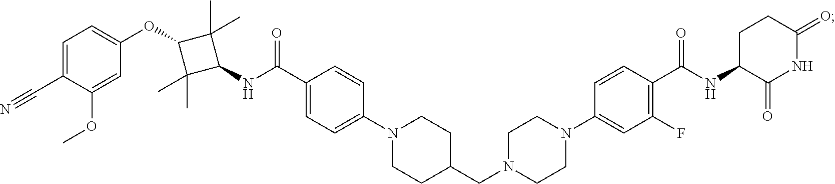 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w microcrystalline cellulose;
7.830% w/w to 11.830% w/w mannitol;
2.000% w/w to 6.000% w/w croscarmellose sodium;
2.000% w/w to 6.000% w/w crospovidone;
0.573% w/w to 0.773% w/w silicon dioxide;
0.250% w/w to 0.750% w/w colloidal silicon dioxide; and
0.100% w/w to 2.000% w/w sodium stearyl fumarate;
   and wherein the extra-granular portion comprises:
1.000% w/w to 4.000% w/w croscarmellose sodium;
0.100% w/w to 1.000% w/w colloidal silicon dioxide; and
0.100% w/w to 1.000% w/w sodium stearyl fumarate.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and Compound A:

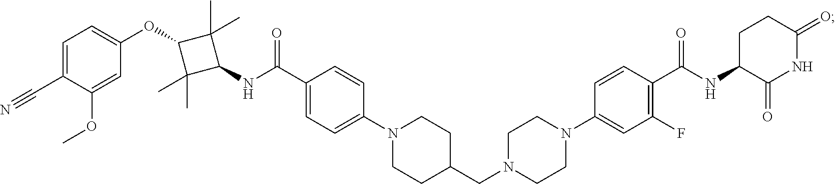 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w microcrystalline cellulose;
7.830% w/w to 11.830% w/w mannitol;
2.000% w/w to 6.000% w/w croscarmellose sodium;
2.000% w/w to 6.000% w/w crospovidone;
0.750% w/w to 2.500% w/w colloidal silicon dioxide; and
0.100% w/w to 2.000% w/w sodium stearyl fumarate;
   and wherein the extra-granular portion comprises:
1.000% w/w to 4.000% w/w croscarmellose sodium;
0.100% w/w to 1.000% w/w colloidal silicon dioxide; and
0.100% w/w to 1.000% w/w sodium stearyl fumarate.

In one aspect, this disclosure pertains to an oral dosage form comprising an intra-granular portion and an extra-granular portion, wherein the intra-granular portion comprises:
65.000% w/w to 68.000% w/w of a solid dispersion comprising a release modifier and a pharmaceutically acceptable salt of Compound A:

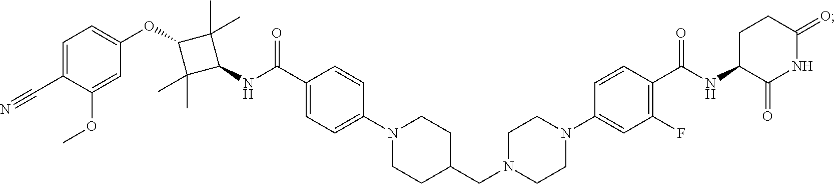 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

7.830% w/w to 11.830% w/w microcrystalline cellulose;
7.830% w/w to 11.830% w/w mannitol;
2.000% w/w to 6.000% w/w croscarmellose sodium;
2.000% w/w to 6.000% w/w crospovidone;
0.573% w/w to 0.773% w/w silicon dioxide;
0.250% w/w to 0.750% w/w colloidal silicon dioxide; and
0.100% w/w to 2.000% w/w sodium stearyl fumarate;
   and wherein the extra-granular portion comprises:
1.000% w/w to 4.000% w/w croscarmellose sodium;
0.100% w/w to 1.000% w/w colloidal silicon dioxide; and
0.100% w/w to 1.000% w/w sodium stearyl fumarate.

In one aspect, this disclosure pertains to an oral dosage form comprising an intra-granular portion and an extra-granular portion, wherein the intra-granular portion comprises:
about 66.667% w/w of a solid dispersion comprising a release modifier and Compound A:

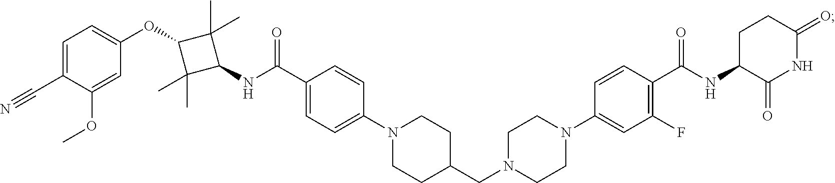 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In one aspect, this disclosure pertains to an oral dosage form comprising an intra-granular portion and an extra-granular portion, wherein the intra-granular portion comprises:
about 66.667% w/w of a solid dispersion comprising a release modifier and a pharmaceutically acceptable salt of Compound A:

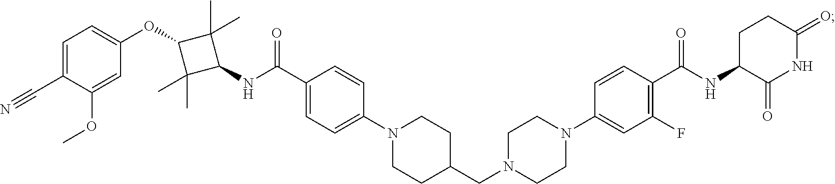 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In one aspect, this disclosure pertains to an oral dosage form comprising an intra-granular portion and an extra-granular portion, wherein the intra-granular portion comprises:
about 66.667% w/w of a solid dispersion comprising a release modifier and a pharmaceutically acceptable salt of Compound A:

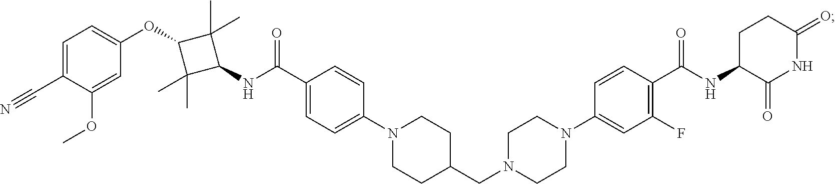 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 10.995% w/w microcrystalline cellulose;
about 3.665% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 5.000% w/w microcrystalline cellulose;
about 2.500% w/w croscarmellose sodium;
about 0.500% w/w colloidal silicon dioxide; and
about 0.500% w/w sodium stearyl fumarate.

In some embodiments, the oral dosage form (e.g., tablet) further comprises a buffer and/or an antioxidant.

In some embodiments, the amount of Compound A, or a pharmaceutically acceptable salt thereof, in the oral dosage form is about 5 mg, about 10 mg, about 25 mg, about 40 mg, about 60 mg, about 75 mg, about 80 mg, about 100 mg, about 120 mg, about 125 mg, or about 150 mg, or an equivalent amount of a pharmaceutically acceptable salt of Compound A.

In some embodiments, the amount of Compound A, or a pharmaceutically acceptable salt thereof, in the oral dosage form is 5 mg, 10 mg, 25 mg, 40 mg, 80 mg, 100 mg, or 120 mg, or an equivalent amount of a pharmaceutically acceptable salt of Compound A.

In some embodiments, the amount of Compound A, or a pharmaceutically acceptable salt thereof, in the oral dosage form is about 5 mg of Compound A, or an equivalent amount of a pharmaceutically acceptable salt of Compound A.

In some embodiments, the amount of Compound A, or a pharmaceutically acceptable salt thereof, in the oral dosage form is about 10 mg of Compound A, or an equivalent amount of a pharmaceutically acceptable salt of Compound A.

In some embodiments, the amount of Compound A, or a pharmaceutically acceptable salt thereof, in the oral dosage form is about 40 mg of Compound A, or an equivalent amount of a pharmaceutically acceptable salt of Compound A.

In some embodiments, the amount of Compound A, or a pharmaceutically acceptable salt thereof, in the oral dosage form is 40 mg of Compound A, or an equivalent amount of a pharmaceutically acceptable salt of Compound A.

In some embodiments, the amount of Compound A, or a pharmaceutically acceptable salt thereof, in the oral dosage form is about 60 mg of Compound A, or an equivalent amount of a pharmaceutically acceptable salt of Compound A.

In some embodiments, the amount of Compound A, or a pharmaceutically acceptable salt thereof, in the oral dosage form is about 75 mg of Compound A, or an equivalent amount of a pharmaceutically acceptable salt of Compound A.

In some embodiments, the amount of Compound A, or a pharmaceutically acceptable salt thereof, in the oral dosage form is about 100 mg of Compound A, or an equivalent amount of a pharmaceutically acceptable salt of Compound A.

In some embodiments, the amount of Compound A, or a pharmaceutically acceptable salt thereof, in the oral dosage form is 100 mg of Compound A, or an equivalent amount of a pharmaceutically acceptable salt of Compound A.

In some embodiments, the amount of Compound A, or a pharmaceutically acceptable salt thereof, in the oral dosage form is about 120 mg of Compound A, or an equivalent amount of a pharmaceutically acceptable salt of Compound A.

In some embodiments, the amount of Compound A, or a pharmaceutically acceptable salt thereof, in the oral dosage form is 120 mg of Compound A, or an equivalent amount of a pharmaceutically acceptable salt of Compound A.

In some embodiments, the amount of Compound A, or a pharmaceutically acceptable salt thereof, in the oral dosage form is about 125 mg of Compound A, or an equivalent amount of a pharmaceutically acceptable salt of Compound A.

In some embodiments, the amount of Compound A, or a pharmaceutically acceptable salt thereof, in the oral dosage form is about 150 mg of Compound A, or an equivalent amount of a pharmaceutically acceptable salt of Compound A.

In some embodiments, the amount of Compound A in the oral dosage form is about 5 mg, about 10 mg, about 25 mg, about 40 mg, about 60 mg, about 75 mg, about 80 mg, about 100 mg, about 120 mg, about 125 mg, or about 150 mg.

In some embodiments, the amount of Compound A in the oral dosage form is 5 mg, 10 mg, 25 mg, 40 mg, 80 mg, 100 mg, or 120 mg.

In some embodiments, the amount of Compound A in the oral dosage form is about 5 mg.

In some embodiments, the amount of Compound A in the oral dosage form is about 10 mg.

In some embodiments, the amount of Compound A in the oral dosage form is about 40 mg.

In some embodiments, the amount of Compound A in the oral dosage form is 40 mg.

In some embodiments, the amount of Compound A in the oral dosage form is about 60 mg.

In some embodiments, the amount of Compound A in the oral dosage form is about 75 mg.

In some embodiments, the amount of Compound A in the oral dosage form is about 100 mg.

In some embodiments, the amount of Compound A in the oral dosage form is 100 mg.

In some embodiments, the amount of Compound A in the oral dosage form is about 120 mg.

In some embodiments, the amount of Compound A in the oral dosage form is 120 mg.

In some embodiments, the amount of Compound A in the oral dosage form is about 125 mg.

In some embodiments, the amount of Compound A in the oral dosage form is about 150 mg.

In some embodiments, for any of the oral dosage forms of the disclosure, the release modifier is hydroxypropyl methylcellulose acetate succinate (HPMCAS-M), hydroxypropyl methylcellulose (HPMC), a copolymer of methacrylic acid and ethyl acrylate, or a poloxamer.

In some embodiments, for any of the oral dosage forms of the disclosure, the release modifier is selected from one or more of the following: copovidone, polyvinyl alcohol, polyvinylpyrrolidone, tocofersolan (vitamin E TPGS), and hydroxypropyl methylcellulose acetate succinate (e.g., HPMCAS-L, HPMCAS-M, and HPMCAS-H).

In some embodiments, for any of the oral dosage forms of the disclosure, the release modifier is selected from one or more of the following: copovidone, polyvinyl alcohol, polyvinylpyrrolidone, tocofersolan (vitamin E TPGS), hydroxypropyl methylcellulose acetate succinate (e.g., HPMCAS-L, HPMCAS-M, and HPMCAS-H), polysorbates (e.g., polysorbate 20, polysorbate 40, polysorbate 60, and polysorbate 80), sorbitan esters (e.g., Span 65, Span 60, and Span 20), and sodium dodecyl sulfate.

In some embodiments, for any of the oral dosage forms of the disclosure, the release modifier is hydroxypropyl methylcellulose acetate succinate (HPMCAS-M).

In some embodiments, for any of the oral dosage forms of the disclosure, the ratio of Compound A, or a pharmaceutically acceptable salt of Compound A, to the release modifier in the solid dispersion is about 3:1 (w/w) to about 1:6 (w/w).

In some embodiments, for any of the oral dosage forms of the disclosure, the ratio of Compound A, or a pharmaceutically acceptable salt of Compound A, to the release modifier in the solid dispersion is about 1:1 (w/w) to about 1:3 (w/w).

In some embodiments, for any of the oral dosage forms of the disclosure, the ratio of Compound A, or a pharmaceutically acceptable salt of Compound A, to the release modifier in the solid dispersion is about 1:1 (w/w).

In some embodiments, for any of the oral dosage forms of the disclosure, the ratio of Compound A, or a pharmaceutically acceptable salt of Compound A, to the release modifier in the solid dispersion is about 1:3 (w/w).

In one aspect, this disclosure pertains to an oral dosage form comprising an intra-granular portion and an extra-granular portion, wherein the intra-granular portion comprises:
about 66.667% w/w of a solid dispersion comprising a release modifier that is HPMCAS-M and Compound A:

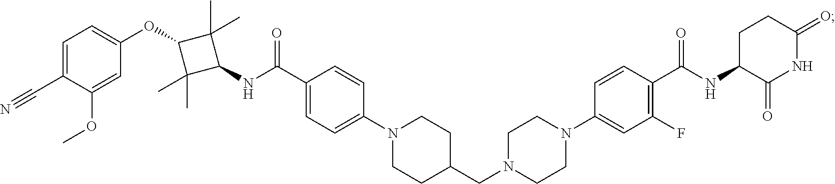 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In one aspect, this disclosure pertains to an oral dosage form comprising an intra-granular portion and an extra-granular portion, wherein the intra-granular portion comprises:
about 66.667% w/w of a solid dispersion comprising a release modifier that is HPMCAS-M and a pharmaceutically acceptable salt of Compound A:

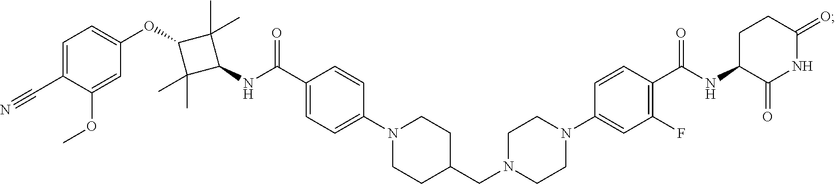 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
   and wherein the extra-granular portion comprises:
about 2.500% w/w croscarmellose sodium;
about 0.500% w/w colloidal silicon dioxide; and
about 0.500% w/w sodium stearyl fumarate.

In some embodiments, for any of the oral dosage forms of the disclosure, where the release modifier that is HPMCAS-M, the ratio of Compound A, or a pharmaceutically acceptable salt of Compound A, to the HPMCAS-M in the solid dispersion is about 3:1 (w/w) to about 1:6 (w/w).

In some embodiments, for any of the oral dosage forms of the disclosure, where the release modifier that is HPMCAS-M, the ratio of Compound A, or a pharmaceutically acceptable salt of Compound A, to the HPMCAS-M in the solid dispersion is about 1:1 (w/w) to about 1:3 (w/w).

In some embodiments, for any of the oral dosage forms of the disclosure, where the release modifier that is HPMCAS-M, the ratio of Compound A, or a pharmaceutically acceptable salt of Compound A, to the HPMCAS-M in the solid dispersion is about 1:1 (w/w).

In some embodiments, for any of the oral dosage forms of the disclosure, where the release modifier that is HPMCAS-M, the ratio of Compound A, or a pharmaceutically acceptable salt of Compound A, to the HPMCAS-M in the solid dispersion is about 1:3 (w/w).

In some embodiments, the formulations of the disclosure are tablets.

In some embodiments, the formulations of the disclosure are formulated for instant release. In some embodiments, the formulations of the disclosure are tablets formulated for instant release. In some embodiments, instant release is synonymous with immediate release.

In some embodiments, the amount of Compound A in the oral dosage form or tablet is from about 5 to about 1000 mg.

In some embodiments, the amount of Compound A in the oral dosage form or tablet is from about 5 to about 500 mg.

In some embodiments, the amount of Compound A in the oral dosage form or tablet is from about 5 to about 450 mg.

In some embodiments, the amount of Compound A in the oral dosage form or tablet is from about 5 to about 75 mg.

In some embodiments, the amount of Compound A in the oral dosage form or tablet is from about 25 to about 400 mg.

In some embodiments, the amount of Compound A in the oral dosage form or tablet is from about 25 to about 75 mg.

In some embodiments, the amount of Compound A in the oral dosage form or tablet is from about 35 to about 350 mg.

In some embodiments, the amount of Compound A in the oral dosage form or tablet is from about 35 to about 75 mg.

In some embodiments, the amount of Compound A in the oral dosage form or tablet is from about 40 to about 350 mg.

In some embodiments, the amount of Compound A in the oral dosage form or tablet is from about 40 to about 75 mg.

In some embodiments, the amount of Compound A in the oral dosage form or tablet is from about 50 to about 300 mg.

In some embodiments, the amount of Compound A in the oral dosage form or tablet is from about 50 to about 75 mg.

In some embodiments, the amount of Compound A in the oral dosage form or tablet is from about 75 to about 300 mg.

In some embodiments, the amount of Compound A in the oral dosage form or tablet is from about 100 to about 300 mg.

In some embodiments, the amount of Compound A in the oral dosage form or tablet is from about 150 to about 300 mg.

In some embodiments, the amount of Compound A in the tablet is from about 100 to about 250 mg.

In some embodiments, the amount of Compound A in the oral dosage form or tablet is about 100 mg.

In some embodiments, the amount of Compound A in the oral dosage form or tablet is about 150 mg.

In some embodiments, the amount of Compound A in the oral dosage form or tablet is about 200 mg.

In some embodiments, the amount of Compound A in the oral dosage form or tablet is about 250 mg.

In some embodiments, the amount of Compound A in the oral dosage form or tablet is about 300 mg.

In some embodiments, the amount of Compound A in the oral dosage form or tablet is about 350 mg.

In some embodiments, the amount of Compound A in the oral dosage form or tablet is about 400 mg.

In some embodiments, the formulation is a tablet that comprises Compound A and/or a pharmaceutically acceptable salt of Compound A.

In some embodiments, the formulation (e.g., tablet) comprises a solid dispersion of Compound A or a pharmaceutically acceptable salt of Compound A.

In some embodiments, the formulation (e.g., tablet) comprises a solid dispersion of Compound A.

In some embodiments, the solid dispersion of Compound A, or a pharmaceutically acceptable salt of Compound A, is an amorphous solid dispersion.

In some embodiments, the solid dispersion of Compound A, or a pharmaceutically acceptable salt Compound A, comprises one or more substances that controls the release of Compound A from the dosage form, i.e., a release modifier.

In some embodiments, the solid dispersion of Compound A, or a pharmaceutically acceptable salt Compound A, comprises one release modifier.

In some embodiments, the solid dispersion of Compound A, or a pharmaceutically acceptable salt Compound A, comprises two release modifiers.

In some embodiments, the solid dispersion of Compound A, or a pharmaceutically acceptable salt Compound A, comprises three or more release modifiers.

In some embodiments, the release modifier is hydroxypropyl methylcellulose acetate succinate (HPMCAS-M).

In some embodiments, the release modifier is hydroxypropyl methylcellulose (HPMC).

In some embodiments, the release modifier is an acrylic polymer.

In some embodiments, the release modifier is a copolymer of methacrylic acid and ethyl acrylate (e.g., Eudragit L100).

In some embodiments, the release modifier is a block copolymer comprising a polyoxypropylene core and two polyoxyethylene side blocks.

In some embodiments, the release modifier is a poloxamer (e.g., Poloxamer 407).

In some embodiments, the release modifier is polyvinyl alcohol.

In some embodiments, the release modifier is polyvinylpyrrolidone.

In some embodiments, the release modifier is tocofersolan (vitamin E TPGS).

In some embodiments, the release modifier comprises both polyvinylpyrrolidone and tocofersolan (vitamin E TPGS).

In some embodiments, the ratio of Compound A, or a pharmaceutically acceptable salt thereof, to the release modifier in the solid dispersion is about 3:1 (w/w) to about 1:6 (w/w).

In some embodiments, the ratio of Compound A, or a pharmaceutically acceptable salt thereof, to the release modifier in the solid dispersion is about 1:1 (w/w) to about 1:3 (w/w).

In some embodiments, the ratio of Compound A to the release modifier in the solid dispersion is about 3:1 (w/w) to about 1:6 (w/w).

In some embodiments, the ratio of Compound A to the release modifier in the solid dispersion is about 1:1 (w/w) to about 1:3 (w/w).

In some embodiments, the ratio of Compound A, or a pharmaceutically acceptable salt thereof, to the release modifier in the solid dispersion is about 3:1 (w/w).

In some embodiments, the ratio of Compound A to the release modifier in the solid dispersion is about 3:1 (w/w).

In some embodiments, the ratio of Compound A, or a pharmaceutically acceptable salt thereof, to the release modifier in the solid dispersion is about 2:1 (w/w).

In some embodiments, the ratio of Compound A to the release modifier in the solid dispersion is about 2:1 (w/w).

In some embodiments, the ratio of Compound A, or a pharmaceutically acceptable salt thereof, to the release modifier in the solid dispersion is about 1:1 (w/w).

In some embodiments, the ratio of Compound A to the release modifier in the solid dispersion is about 1:1 (w/w).

In some embodiments, the ratio of Compound A, or a pharmaceutically acceptable salt thereof, to the release modifier in the solid dispersion is about 1:2 (w/w).

In some embodiments, the ratio of Compound A to the release modifier in the solid dispersion is about 1:2 (w/w).

In some embodiments, the ratio of Compound A, or a pharmaceutically acceptable salt thereof, to the release modifier in the solid dispersion is about 1:3 (w/w).

In some embodiments, the ratio of Compound A to the release modifier in the solid dispersion is about 1:3 (w/w).

In some embodiments, the ratio of Compound A, or a pharmaceutically acceptable salt thereof, to the release modifier in the solid dispersion is about 1:4 (w/w).

In some embodiments, the ratio of Compound A to the release modifier in the solid dispersion is about 1:4 (w/w).

In some embodiments, the ratio of Compound A, or a pharmaceutically acceptable salt thereof, to the release modifier in the solid dispersion is about 1:5 (w/w).

In some embodiments, the ratio of Compound A to the release modifier in the solid dispersion is about 1:5 (w/w).

In some embodiments, the ratio of Compound A, or a pharmaceutically acceptable salt thereof, to the release modifier in the solid dispersion is about 1:6 (w/w).

In some embodiments, the ratio of Compound A to the release modifier in the solid dispersion is about 1:6 (w/w).

In some embodiments, the solid dispersion is prepared by spray-drying a solution of Compound A, or a pharmaceutically acceptable salt thereof, one or more release modifiers, and one or more solvents.

Carriers include pharmaceutically acceptable excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject. Examples include, but are not limited to, calcium carbonate, calcium phosphate, various sugars, starches, cellulose derivatives, gelatin, and polymers such as polyethylene glycols.

As used herein, a “polysaccharide” includes, but is not limited to, glycogen, starches (e.g., potato, wheat, tapioca, corn, and rice starches), celluloses (e.g., microcrystalline cellulose), cellulose ether derivatives (e.g., methylcellulose, ethylcellulose, hydroxyethylmethylcellulose, hydroxypropylcellulose, and carboxymethylcellulose), cellulose ester derivatives (e.g., cellulose acetate, cellulose acetate phthalate, cellulose acetate trimellitate, cellulose hydroxypropylmethylphthalate, and cellulose hydroxypropylmethylphthalate acetate succinate), chitin, and chondroitin sulfate.

As used herein, a “sugar alcohol” includes, but is not limited to, sorbitol, mannitol, erythritol, lactitol, isomalt, maltitol, xylitol, and hydrogenated starch hydrolysates.

Fillers include, but are not limited to, mannitol, sucrose, sorbitol, xylitol, microcrystalline cellulose, lactose, silicic acid, silicified microcrystalline cellulose, hydroxypropyl methylcellulose, hydroxypropyl cellulose, starch, pullulan and fast dissolving carbohydrates such as Pharmaburst™ fast disintegrating tablets, mixtures thereof, and the like. For examples of fast-dissolving carbohydrates see, e.g., U.S. Pat. No. 8,617,588, which is incorporated herein by reference. In an embodiment, the filler is a polysaccharide (e.g., microcrystalline cellulose) and/or a sugar alcohol (e.g., mannitol). In another embodiment, the filler is a polysaccharide (e.g., microcrystalline cellulose) and a sugar alcohol (e.g., mannitol). In yet another embodiment, the filler is a polysaccharide (e.g., microcrystalline cellulose). In still another embodiment, the filler is a sugar alcohol (e.g., mannitol).

Surfactants include, but are not limited to, non-ionic, anionic, cationic, amphoteric or zwitterionic surfactants. Examples of suitable non-ionic surfactants include ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™; alkyl polyglucosides; stearol ethoxylates; alkyl polyglycosides. Examples of suitable anionic surfactants include alkylether sulfates; alkylether carboxylates; alkyl benzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates. Examples of suitable cationic surfactants include fatty amine salts; fatty diamine salts; quaternary ammonium compounds; phosphonium surfactants; sulfonium surfactants; sulfoxonium surfactants. Examples of suitable zwitterionic surfactants include N-alkyl derivatives of amino acids (such as glycine, betaine, aminopropionic acid); imidazoline surfactants; amine oxides; amidobetaines. Non-limiting examples of a surfactant that can be used in the ospemifene solid dispersions, include, for example. Tween 20, Tween 80, Span 20, Span 80, sodium docusate (e.g., AOT), sodium lauryl sulfate, and poloxamers (e.g., poloxamer 407, Kolliphor® EL, Pluronic F68). Poloxamers are also known by the trade names Synperonics®, Pluronics®, and Kolliphor®/Cremophor®.

Diluents include, but are not limited to, carbohydrates such as monosaccharides like glucose, oligosaccharides like sucrose and lactose (including anhydrous lactose and lactose monohydrate), starch such as maize starch, potato starch, rice starch and wheat starch, pregelatinized starch, calcium hydrogen phosphate, and sugar alcohols like sorbitol, mannitol, erythritol, and xylitol.

Sweeteners include, but are not limited to, sucrose, high fructose corn syrup, fructose, glucose, aspartame, acesulfame K, sucralose, cyclamate, sodium saccharin, neotame, rebaudioside A, and other stevia-based sweeteners.

Disintegrants include, but are not limited to, sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkyl substituted hydroxypropyl cellulose, hydroxylpropyl starch, low-substituted hydroxypropylcellulose, polacrilin potassium, starch, pregelatinized starch, sodium alginate, magnesium aluminum silicate, polacrilin potassium, povidone, sodium starch glycolate, mixtures thereof, and the like. In an embodiment, the disintegrant is a carboxymethylcellulose derivative (e.g., croscarmellose sodium) and/or crospovidone. In another embodiment, the disintegrant is croscarmellose sodium and crospovidone. In yet another embodiment, the disintegrant is croscarmellose sodium. In still another embodiment, the disintegrant is crospovidone.

Carboxymethylcellulose derivatives include, but are not limited to, sodium carboxymethyl cellulose, calcium carboxymethyl cellulose, and croscarmellose sodium.

Binders include, but are not limited to, hydroxypropylmethylcellulose (HPMC), hydroxypropyl cellulose (HPC), povidone, copovidone, methylcellulose, powdered acacia, gelatin, gum arabicum, guar gum, carbomer such as carbopol, and polymethacrylates.

Lubricants include, but are not limited to, calcium stearate, glyceryl monostearate, glyceryl behenate, glyceryl palmitostearate, hexagonal boron nitride, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, mixtures thereof, and the like.

Glidants include, but are not limited to, silicon dioxide, colloidal silicon dioxide, calcium silicate, magnesium silicate, magnesium trisilicate, talc, starch, mixtures thereof, and the like.

Antioxidants include, but are not limited to, potassium metabisulfite, sodium metabisulfite, ascorbic acid (and derivates), butylated hydroxyanisole, butylated hydroxytoluene, tocopherol, and cysteine.

Buffers include, but are not limited to, acetate buffers, citrate buffers, glutamate buffers, maleate buffers, phosphate buffers, histidine buffers, borate buffers, and carbonate buffers.

Flavors include, but are not limited to, menthol, peppermint oil, peppermint spirit, vanillin, and almond oil.

Examples of release modifiers include, but are not limited to, hydroxypropyl methylcellulose (HPMC), hydroxypropyl methylcellulose acetate succinate (HPMCAS-M), polyvinyl alcohol, ethyl cellulose, acrylic polymers (e.g., a methacrylic polymer), hydroxypropyl cellulose, starches, gums, cellulose ethers, protein derived materials, nylon, acrylic resins, polylactic acid, polyvinylchloride, polyvinylpyrrolidones, a block copolymer comprising a polyoxypropylene core and two polyoxyethylene side blocks (e.g., a poloxamer), and cellulose acetate phthalate. In an embodiment, the acrylic polymer is a copolymer of methacrylic acid and ethyl acrylate (e.g., Eudragit L100). In another embodiment, the poloxamer is Poloxamer 407.

Some excipients can act as a binder, filler and/or a disintegrant, depending on their content in the blend, coexisting excipients and how and when they are formulated in the formulation. It is completely in the purview of a skilled artisan to understand the role of a pharmaceutical excipient in the composition. They will be able to assess the role of the excipient in the pharmaceutical composition or the dosage form based on the desired characteristics, such as volume, hardness, dissolution profile etc, of the pharmaceutical composition or the dosage form.

In some embodiments, the pharmaceutical formulations of the disclosure are uncoated.

In some embodiments, the pharmaceutical formulations of the disclosure are coated.

In some embodiments, the pharmaceutical formulations of the disclosure are uncoated tablets.

In some embodiments, the pharmaceutical formulations of the disclosure are coated tablets.

In some embodiments, the pharmaceutical formulations of the disclosure are coated tablets with a film that comprises a polymer, a plasticizer, a pigment, or combinations thereof.

In an aspect, this disclosure pertains to an oral dosage form (e.g., tablet) selected from Tables 1A, 1B, 1C, 1D, 1E, 1F, 1G, 1H, and 1I.

### Methods of Manufacturing

In one aspect, this disclosure pertains to a method of manufacturing a tablet of the disclosure, wherein the method comprises:
(a) blending the intragranular ingredients to create a pre-blend;
(b) granulating the pre-blend to provide a milled blend;
(c) blending the milled blend with the extragranular ingredients to provide a final blend;
(d) compressing the final blend to provide the tablet or oral dosage form; and
(e) optionally, coating the tablet or oral dosage form.

In embodiments, the method comprises:
(e) coating the tablet or oral dosage form.

In one aspect, this disclosure pertains to a method of manufacturing a tablet of the disclosure, wherein the method comprises:
(a) dissolving Compound A, or pharmaceutically acceptable salt thereof, and dissolving and/or suspending appropriate excipients to provide a solution or a suspension;
(b) spray-drying the solution or suspension to provide a wet spray-dried dispersion;
(c) drying the wet spray-dried dispersion to provide a combination;
(d) compressing the combination into tablets or blending the combination with appropriate excipients and then compressing into tablets;
(e) optionally, coating the tablets.

In one aspect, this disclosure pertains to a method of manufacturing a combination comprising an amorphous form of Compound A and a release modifier, wherein the method comprises:
(a) dissolving Compound A, or a pharmaceutically acceptable salt of Compound A, and the release modifier in a solvent to provide a solution;
(b) spray-drying the solution to provide a wet spray-dried dispersion; and
(c) drying the wet spray-dried dispersion to provide the combination.

In one aspect, this disclosure pertains to a method of manufacturing a combination comprising an amorphous form of Compound A and a release modifier, wherein the method comprises:
(a) dissolving Compound A, or a pharmaceutically acceptable salt of Compound A, and the release modifier in a solvent to provide a solution;
(b) granulating the solution by spray-granulation to provide a wet spray-granulated dispersion; and
(c) drying the wet spray-granulated dispersion to provide the combination.

In embodiments, the solvent comprises DCM and methanol.

In embodiments, the solvent comprises DCM and methanol in a ratio of about 20:1 (w/w) to about 1:1 (w/w), e.g., 9:1 (w/w).

In embodiments, the solvent comprises DCM and methanol in a ratio of about 20:1 (v/v) to about 1:1 (v/v), e.g., 9:1 (v/v).

In one aspect, this disclosure pertains to a method of manufacturing a combination comprising an amorphous form of Compound A and a release modifier, wherein the method comprises:
(a) dissolving and/or melting Compound A, or a pharmaceutically acceptable salt of Compound A, and the release modifier at elevated temperature to provide a hot solution and/or hot melt; and
(b) extruding the solution and/or hot melt by extrusion to provide an extruded dispersion.

In an embodiment of any of the above aspects, the solvent comprises, but is not limited to, one or more of dichloromethane (DCM), methanol, tetrahydrofuran (THF), acetone, water, acetone, ethanol, formic acid, and acetic acid.

In an embodiment of any of the above aspects, the solvent comprises DCM and methanol.

In an embodiment of any of the above aspects, the solvent comprises DCM and methanol in a ratio of about 20:1 (w/w) to about 1:1 (w/w), e.g., 9:1 (w/w).

In an embodiment of any of the above aspects, the solvent comprises THF and water in a ratio of about 70:30 (w/w) to about 99.9:0.01 (w/w), e.g., 98:2 (w/w).

In an embodiment of any of the above aspects, the solvent comprises acetone and acetic acid in a ratio of about 70:30 (w/w) to about 99.9:0.01 (w/w), e.g., 80:20 (w/w).

In an embodiment of any of the above aspects, the solvent comprises acetone and formic acid in a ratio of about 70:30 (w/w) to about 99.9:0.01 (w/w), e.g., 95:5 (w/w).

In an embodiment of any of the above aspects, the solids concentration dissolved in the solvent or solvent mixtures comprises acetone and formic acid is about 5% (w/w) to about 20% (w/w), e.g., 8% (w/w).

In an embodiment of any of the above aspects, the solvent or solvent mixture comprises counter ions for pH adjustment and/or to improve the solubilization of Compound A is about 0.001% w/w to 5% w.w, e.g. 1% w/w.

### Methods of Treatment

In one aspect, the present disclosure pertains to a method of treating cancer comprising administering to a subject in need thereof a therapeutically effective amount of a dosage form or tablet disclosed herein. In some embodiments, the cancer is prostate cancer.

In some embodiments, the prostate cancer is metastatic prostate cancer. In some embodiments, the prostate cancer is castrate-resistant prostate cancer. In some embodiments, the prostate cancer is metastatic castrate-resistant prostate cancer (mCRPC). In some embodiments, the prostate cancer is progressive mCRPC. In some embodiments, the prostate cancer is castrate-sensitive prostate cancer. In some embodiments, the prostate cancer is metastatic castrate-sensitive prostate cancer. In some embodiments, the prostate cancer is prostate cancer naïve to novel hormonal agents (NHA). In some embodiments, the prostate cancer is metastatic prostate cancer naïve to novel hormonal agents (NHA). In some embodiments, the prostate cancer is castrate-resistant prostate cancer naïve to novel hormonal agents (NHA). In some embodiments, the prostate cancer is castrate-sensitive prostate cancer naïve to novel hormonal agents (NHA). In some embodiments, the prostate cancer is metastatic castrate-resistant prostate cancer naïve to novel hormonal agents (NHA). In some embodiments, the prostate cancer is metastatic castrate-sensitive prostate cancer naïve to novel hormonal agents (NHA).

In one aspect, treating cancer results in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its volume prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means.

In another aspect, treating cancer results in a decrease in tumor growth rate. Preferably, after treatment, tumor growth rate is reduced by at least 5% relative to growth rate prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured by measuring an increase in the diameter of a tumor after a prior tumor shrinkage that followed treatment. In another preferred aspect, a decrease in tumor regrowth is indicated by failure of tumors to reoccur after treatment has stopped.

The therapeutically effective amount of the dosage forms or tablets of the disclosure can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Dosage forms or tablets that exhibit large therapeutic indices are preferred. The dosage may vary within this range depending upon the dosage form employed, sensitivity of the patient, and the route of administration.

Dosage and administration are adjusted to provide sufficient levels of Compound A or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

### Equivalents and Scope

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Exemplary Preparation of Tablets/Capsules

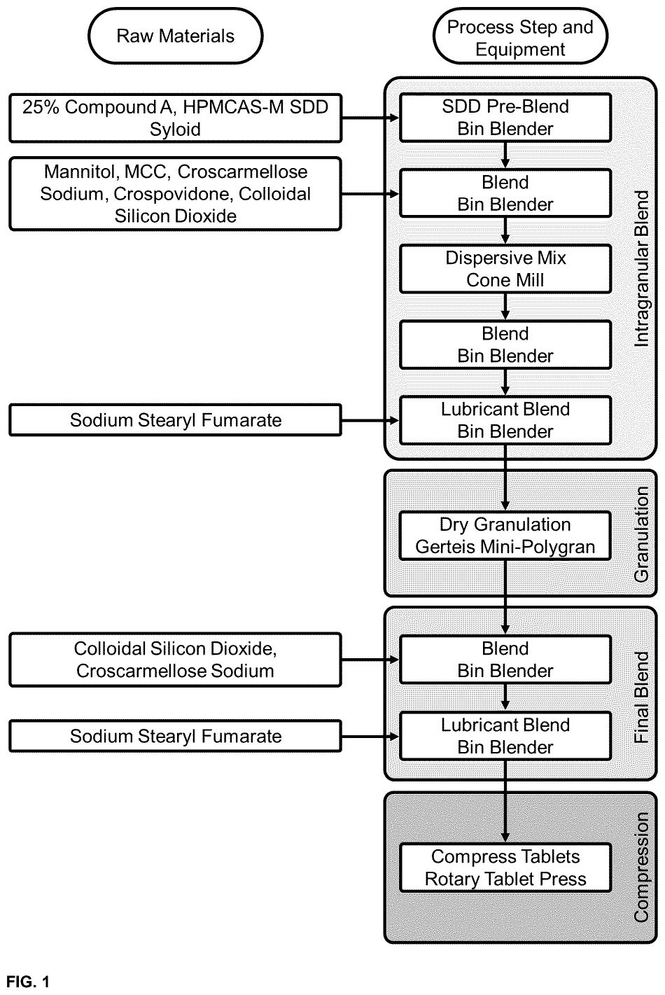

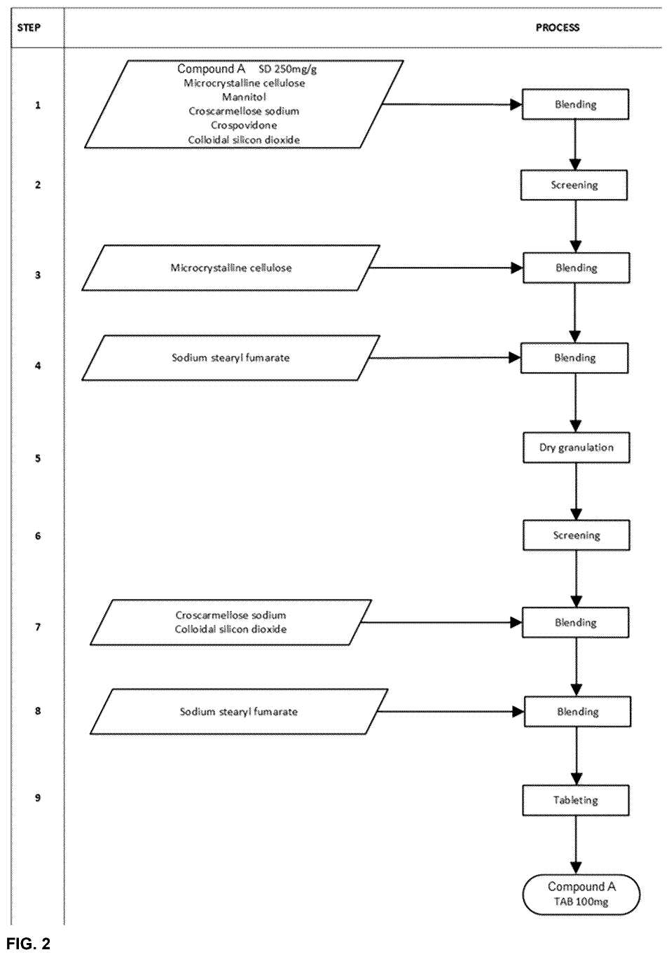

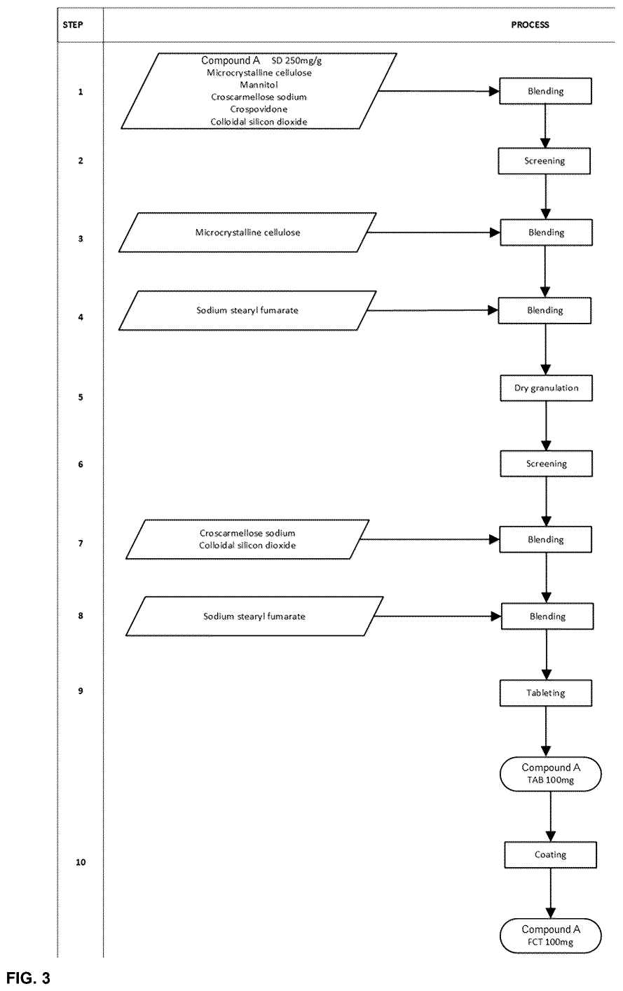

Tablets comprising Compound A were prepared following the procedures described in FIG. 1, FIG. 2, and FIG. 3. The compositions of the tablets are summarized in Tables 1A, 1B, 1C, 1D, 1E, 1F, 1G, 1H, and 1I.

TABLE 1A

40 mg Tablet*

| | | |
|---|---|---|
| Ingredient | % w/w | mg |

Intragranular

| | | |
|---|---|---|
| Solid Dispersion of Compound A | 66.667 | 160.000 |
| and HPMCAS-M (1:3 w/w) | | |
| Silicon dioxide | 0.673 | 1.616 |
| Microcrystalline cellulose (MCC) | 9.830 | 23.592 |
| Mannitol | 9.830 | 23.592 |
| Croscarmellose sodium | 4.000 | 9.600 |
| Crospovidone | 4.000 | 9.600 |
| Colloidal silicon dioxide | 0.500 | 1.200 |
| Sodium stearyl fumarate | 1.000 | 2.400 |
| Intragranular Total | 96.500 | 231.600 |

Extragranular

| | | |
|---|---|---|
| Croscarmellose sodium | 2.500 | 6.000 |
| Colloidal silicon dioxide | 0.500 | 1.200 |
| Sodium stearyl fumarate | 0.500 | 1.200 |
| Total | 100.000 | 240.000 |

*refers to the amount of Compound A in the tablet

TABLE 1B

100 mg Tablet*

| | | |
|---|---|---|
| Ingredient | % w/w | mg |

Intragranular

| | | |
|---|---|---|
| Solid Dispersion of Compound A | 66.667 | 400.000 |
| and HPMCAS-M (1:3 w/w) | | |
| Silicon dioxide | 0.673 | 4.038 |
| Microcrystalline cellulose | 9.830 | 58.981 |
| Mannitol | 9.830 | 58.981 |
| Croscarmellose sodium | 4.000 | 24.000 |
| Crospovidone | 4.000 | 24.000 |
| Colloidal silicon dioxide | 0.500 | 3.000 |
| Sodium stearyl fumarate | 1.000 | 6.000 |
| Intragranular Total | 96.500 | 579.000 |

Extragranular

| | | |
|---|---|---|
| Croscarmellose sodium | 2.500 | 15.000 |
| Colloidal silicon dioxide | 0.500 | 3.000 |
| Sodium stearyl fumarate | 0.500 | 3.000 |
| Total | 100.000 | 600.000 |

*refers to the amount of Compound A in the tablet

TABLE 1C

120 mg Tablet*

| | | |
|---|---|---|
| Ingredient | % w/w | mg |

Intragranular

| | | |
|---|---|---|
| Solid Dispersion of Compound A | 66.667 | 480.000 |
| and HPMCAS-M (1:3 w/w) | | |
| Silicon dioxide | 0.673 | 4.848 |
| Microcrystalline cellulose | 9.830 | 70.776 |
| Mannitol | 9.830 | 70.776 |
| Croscarmellose sodium | 4.000 | 28.800 |
| Crospovidone | 4.000 | 28.800 |
| Colloidal silicon dioxide | 0.500 | 3.600 |
| Sodium stearyl fumarate | 1.000 | 7.200 |
| Intragranular Total | 96.500 | 694.800 |

Extragranular

| | | |
|---|---|---|
| Croscarmellose sodium | 2.500 | 18.000 |
| Colloidal silicon dioxide | 0.500 | 3.600 |
| Sodium stearyl fumarate | 0.500 | 3.600 |
| Total | 100.000 | 720.000 |

*refers to the amount of Compound A in the tablet

TABLE 1D

100 mg Tablet*

| | | |
|---|---|---|
| Ingredient | % w/w | mg |

Intragranular

| | | |
|---|---|---|
| Solid Dispersion of Compound A | 66.667 | 400.000 |
| and HPMCAS-M (1:3 w/w) | | |
| Microcrystalline cellulose | 9.830 | 58.981 |
| Mannitol | 9.830 | 58.981 |
| Croscarmellose sodium | 4.000 | 24.000 |
| Crospovidone | 4.000 | 24.000 |
| Colloidal silicon dioxide | 1.173 | 7.038 |
| Sodium stearyl fumarate | 1.000 | 6.000 |
| Intragranular Total | 96.500 | 579.000 |

Extragranular

| | | |
|---|---|---|
| Croscarmellose sodium | 2.500 | 15.000 |
| Colloidal silicon dioxide | 0.500 | 3.000 |
| Sodium stearyl fumarate | 0.500 | 3.000 |
| Total | 100.000 | 600.000 |

*refers to the amount of Compound A in the tablet

TABLE 1E

40 mg film-coated Tablet*

| | | |
|---|---|---|
| Ingredient | % w/w | mg |

Intragranular

| | | |
|---|---|---|
| Solid Dispersion of Compound A | 64.412 | 160.000 |
| and HPMCAS-M (1:3 w/w) | | |
| Microcrystalline cellulose | 9.498 | 23.592 |
| Mannitol | 9.498 | 23.592 |
| Croscarmellose sodium | 3.865 | 9.600 |
| Crospovidone | 3.865 | 9.600 |
| Colloidal silicon dioxide | 1.134 | 2.816 |
| Sodium stearyl fumarate | 0.966 | 2.400 |
| Intragranular Total | 93.240 | 231.600 |

Extragranular

| | | |
|---|---|---|
| Croscarmellose sodium | 2.415 | 6.000 |
| Colloidal silicon dioxide | 0.483 | 1.200 |
| Sodium stearyl fumarate | 0.483 | 1.200 |
| Total Core | 96.620 | 240.000 |
| Polyvinyl alcohol-based film-coat | 3.382 | 8.400 |
| with titanium dioxide, talcum, | | |
| macrogol and ferric oxides. | | |
| Total | 100.000 | 248.400 |

*refers to the amount of Compound A in the tablet

TABLE 1F

75 mg film-coated Tablet*

| | | |
|---|---|---|
| Ingredient | % w/w | mg |

Intragranular

| | | |
|---|---|---|
| Solid Dispersion of Compound A | 64.412 | 300.000 |
| and HPMCAS-M (1:3 w/w) | | |
| Microcrystalline cellulose | 9.498 | 44.235 |
| Mannitol | 9.498 | 44.235 |
| Croscarmellose sodium | 3.865 | 18.000 |
| Crospovidone | 3.865 | 18.000 |
| Colloidal silicon dioxide | 1.134 | 5.280 |
| Sodium stearyl fumarate | 0.966 | 4.500 |
| Intragranular Total | 93.240 | 434.250 |

Extragranular

| | | |
|---|---|---|
| Croscarmellose sodium | 2.415 | 11.250 |
| Colloidal silicon dioxide | 0.483 | 2.250 |
| Sodium stearyl fumarate | 0.483 | 2.250 |
| Total Core | 96.620 | 450.000 |
| Polyvinyl alcohol-based film-coat | 3.382 | 15.750 |
| with titanium dioxide, talcum, | | |
| macrogol and ferric oxides. | | |
| Total | 100.000 | 465.750 |

TABLE 1G

100 mg film-coated Tablet*

| | | |
|---|---|---|
| Ingredient | % w/w | mg |

Intragranular

| | | |
|---|---|---|
| Solid Dispersion of Compound A | 64.412 | 400.000 |
| and HPMCAS-M (1:3 w/w) | | |
| Microcrystalline cellulose | 9.498 | 58.981 |
| Mannitol | 9.498 | 58.981 |
| Croscarmellose sodium | 3.865 | 24.000 |
| Crospovidone | 3.865 | 24.000 |
| Colloidal silicon dioxide | 1.134 | 7.038 |
| Sodium stearyl fumarate | 0.966 | 6.000 |
| Intragranular Total | 93.240 | 579.000 |

Extragranular

| | | |
|---|---|---|
| Croscarmellose sodium | 2.415 | 15.000 |
| Colloidal silicon dioxide | 0.483 | 3.000 |
| Sodium stearyl fumarate | 0.483 | 3.000 |
| Total Core | 96.620 | 600.000 |
| Polyvinyl alcohol-based film-coat | 3.382 | 21.000 |
| with titanium dioxide, talcum, | | |
| macrogol and ferric oxides. | | |
| Total | 100.000 | 621.000 |

*refers to the amount of Compound A in the tablet

TABLE 1H

125 mg film-coated Tablet*

| | | |
|---|---|---|
| Ingredient | % w/w | mg |

Intragranular

| | | |
|---|---|---|
| Solid Dispersion of Compound A | 64.412 | 500.000 |
| and HPMCAS-M (1:3 w/w) | | |
| Microcrystalline cellulose | 9.498 | 73.725 |
| Mannitol | 9.498 | 73.725 |
| Croscarmellose sodium | 3.865 | 30.000 |
| Crospovidone | 3.865 | 30.000 |
| Colloidal silicon dioxide | 1.134 | 8.800 |
| Sodium stearyl fumarate | 0.966 | 7.500 |
| Intragranular Total | 93.240 | 723.750 |

Extragranular

| | | |
|---|---|---|
| Croscarmellose sodium | 2.415 | 18.750 |
| Colloidal silicon dioxide | 0.483 | 3.750 |
| Sodium stearyl fumarate | 0.483 | 3.750 |
| Total Core | 96.620 | 750.000 |
| Polyvinyl alcohol-based film-coat | 3.382 | 26.250 |
| with titanium dioxide, talcum, | | |
| macrogol and ferric oxides. | | |
| Total | 100.000 | 776.250 |

*refers to the amount of Compound A in the tablet

TABLE 1I

150 mg Tablet*

| | | |
|---|---|---|
| Ingredient | % w/w | mg |

Intragranular

| | | |
|---|---|---|
| Compound A and HPMCAS-M (1:1 | 66.667 | 300.000 |
| w/w) Spray-Dried Dispersion | | |
| Microcrystalline cellulose | 10.995 | 49.480 |
| Mannitol | 3.665 | 16.490 |
| Croscarmellose sodium | 4.000 | 18.000 |
| Crospovidone | 4.000 | 18.000 |
| Silicon dioxide | 0.673 | 3.030 |
| Colloidal silicon dioxide | 0.500 | 2.250 |
| Sodium stearyl fumarate | 1.000 | 4.500 |
| Intragranular Total | 91.500 | 411.750 |

Extragranular

| | | |
|---|---|---|
| Microcrystalline cellulose | 5.000 | 22.500 |
| Croscarmellose sodium | 2.500 | 11.250 |
| Colloidal silicon dioxide | 0.500 | 2.250 |
| Sodium stearyl fumarate | 0.500 | 2.250 |
| Total | 100.000 | 450.000 |

*refers to the amount of Compound A in the tablet

### Example 2. Exemplary Preparation of Amorphous Form and Dispersion

Spray-drying process development for 25% Compound A, HPMCAS-M resulted in a process that was successfully scaled to pilot-scale. This process used a 5-9 wt % solid solution in 90:10% w/w DCM:Methanol atomized with a Spraying Systems SK 80-16 nozzle at 250-280 g/min liquid feed rate with an outlet temperature of 35-45° C. Secondary drying process development work identified two options: tray drying for 20 h at 45° C. or agitated bed vacuum drying (in a conical dryer) for 12 h at 45° C. Results from an in-process stability study resulted in nominating a solution hold time of up to 48 h at ambient conditions, up to 7 days at 2-8° C. and a solvent-laden spray-dried dispersions hold time of up to 16 days at ambient conditions or 2-8° C. This spray-drying process was successfully scaled to GMP Operations for clinical trial material (CTM) manufacture. Further SDD optimization aimed at increasing the active loading was performed.

The processing parameters for the manufacture of the spray-dried dispersions (SDD) of Compound and HPMCAS-M (1:3, w/w) are provided below in Table 1J.

TABLE 1J
Spray-Drying Process for GMP Manufacturing.

| Parameter | Set point |
|---|---|
| Formulation | 25% Compound A, 75% HPMCAS-M |
| Solvent system | 90:10 DCM:Methanol |
| Nozzle | Spraying Systems SK80-16 |
| Solids loading (wt %) | 6.0 |
| Drying gas feed rate | 85 |
| (acfm) | |
| Liquid feed rate (g/min) | 280 |
| Atomization pressure | 650 |
| (psi) | |
| Drying gas inlet | 95 |
| temperature (° C.) | |
| Spray-dryer outlet | 40 |
| temperature (° C.) | |
| Condenser set point | −20 |
| (° C.) | |
| Solution hold time | 48 h at room temperature or up to 7 days |
| | at 2-8° C. |
| Wet SDD hold time | Up to 16 days at 2-8° C. or room |
| | temperature |
| Secondary drying | 45° C. for 20 h |
| conditions (convection | |
| tray dryer) | |
| Secondary drying | 45° C. for 12 h, with or without 1 wt % Syloid |
| conditions (vacuum | 244FP added to the SDD to enhance flow |
| conical dryer) | |

### Example 3. Exemplary Methods for Characterizing Tablets

### Powder Blend and Granule Density

Bulk and tap densities were measured to evaluate powder and granule properties, to provide an assessment of flow, and to provide comparisons between blend and granulation batches. Bulk and tap densities were measured with guidance from USP <616>, Method I. About 30-40 g samples were weighed and gently added to a 100 mL graduated cylinder. The undisturbed volume was used to calculate bulk density (sample mass/undisturbed volume). Tap density was calculated from the tapped (settled) volume of the sample after testing on a Quantachrome AutoTap (sample mass/settled volume) instrument. The Carr Index (CI) was then calculated to assess flowability as follows:

$$Carr Index ( \% ) = 100 * ( 1 - \frac{bulk density}{tap density} )$$

### Particle Size Distribution by Sieve Analysis

The particle size distribution of the granules and final blends was determined using sieve analysis by placing 30 g of granules on a nest of 8″ round sieves in a RoTap RX-29 sieve shaker for 5 minutes per USP <786>. Masses of each sieve fraction were determined, and the particle size distribution was calculated.

### Ribbon Solid Fraction

Ribbon solid fraction was determined by measuring ribbon pieces in a Micrometrics GeoPyc 1265 with a 40 cm cell. Ribbon samples (15-25% of cell volume) were placed in the cell and envelope density was measured (parameters). Results are the average of three measurements.

### Hardness (Breaking Force) and Tensile Strength

Tablet “hardness”, or breaking force, was assessed via the PTB 511E Hardness Tester. The hardness tester is equipped with a rectangular ram that compresses tablets against a plate until fracture occurs. Round tablets were fractured across their diameter. The breaking force was recorded in kiloponds. Fracture stress, or tensile strength, for flat-faced tablets was determined using the expression below:

$${σ}_{t} = \frac{2 * P}{π * D * t}$$

The tensile strength for round concave was determined using another expression from Pitt et al:

$${σ}_{t} = \frac{10 P}{π {D}^{2} ( 2.84 \frac{t}{D} + 0.126 \frac{t}{W} + 3.15 \frac{W}{D} + 0.01 )} .$$

Lastly, the tensile strength for convex-faced elongated tablets was determined by the modified expression from Pitt et al:

$${σ}_{t} = \frac{2}{3} ( \frac{10 P}{π {D}^{2} ( 2.84 \frac{t}{D} + 0.126 \frac{t}{W} + 3.15 \frac{W}{D} + 0.01 )} ) ,$$

Where σₜ is the fracture stress in MPa, P is the breaking force in N, D is the tablet diameter in mm, and t is the overall tablet thickness in mm. Samples were assessed within an hour off-the-press (OTP) to minimize the variability of uncontrolled relaxation time after compression.

### Disintegration

Disintegration was assessed in close accordance with USP <701> utilizing a Sotax DT2 disintegration tester. 700 mL of 0.01 N HCl at 37° C. (+/−2° C.) was used as the disintegration medium. The disintegration time was recorded as the time tablet material visually passed through the mesh screen at the bottom of the oscillating (29-32 cycles/min) basket apparatus.

### Dissolution

Sink dissolution was measured using dissolution apparatus and HPLC parameters as detailed in the tables below.

TABLE 2
Summary of dissolution experimental parameters.

| Parameter | Value | |
|---|---|---|
| Apparatus | Agilent 708 DS | |
| Dissolution Media | 0.5% SLS in 0.01N HCl (100 mg tablets) | |
| Temperature | 37.0 ± 0.5° | C. |
| Vessel Volume | 900 ± 9 | mL |
| Stir Rate | 50 rpm | (start - 60 min) |
| | 225 rpm | (infinity - spin 60 to 75 min) |
| Sampling Volume | 3 | mL |
| Sampling Time | 10, 30, 45, 60 min | (75 min infinity spin) |
| Points | | |
| Sample Filter | 20 μm | PE filter |

TABLE 3
Rapid quantitation HPLC method.

| | |
|---|---|
| Parameter | Setting |

Sample Preparation

| | |
|---|---|
| Standard Diluent | 10:90 (4:1 Acetonitrile:Dimethyl Sulfoxide):(0.5% |
| | SLS in 0.01N HCl) |
| Sample Diluent | 0.5% SLS in 0.01N HCl (100 mg tablets) |

HPLC Methodology

| Column | Agilent Zorbax 300 Extend-C18, 4.6 × 50 mm, 3.5 μm | |
|---|---|---|
| | (P/N: 765973-902) | |
| Flow Rate | 1 | mL/min |
| Column | 40° | C. |
| Temperature | | |
| Needle Wash | 4:1 ACN:DMSO | |
| Injection Volume | 5 | μL |
| Detection | 292 | nm |
| Wavelength | | |

| Mobile Phase A | | 10 mM Ammonium Acetate in Water, pH 9 | | |
|---|---|---|---|---|
| Mobile Phase B | | Acetonitrile | | |
| | Gradient | Time (min) | % MPA | % MPB |
| | | 0.00 | 60 | 40 |
| | | 1.00 | 60 | 40 |
| | | 4.00 | 5 | 95 |
| | | 7.00 | 5 | 95 |
| | | 7.01 | 60 | 40 |
| | | 10.00 | 60 | 40 |

### Example 4. Powder and Tablet Characterization

### Ribbon and Slug Fraction

The ribbon solid fraction values of the 40 mg, 100 mg, and 120 mg strength batches at 4 kN/cm roll force, the roll force used for the main granulations, were measured to be the same, as illustrated in Table 4.

TABLE 4
Roller compaction roll force and associated ribbon solid fraction.

| | Roll Force | Ribbon Solid |
|---|---|---|
| Lot No. | (kN/cm) | Fraction* |
| 40 mg & 120 mg | 4.0 kN/cm | 0.59 |
| 100 mg | 4.0 kN/cm | 0.59 |

*Absolute density of 1.38 g/cc used for calculating solid fraction

### Granulation and Final Blend Bulk Density, Tapped Density, and Carr Index

The bulk and tap densities of the granulations and final blends of the 40 mg, 100 mg, and 120 mg strength batches are summarized below in Table 5.

The 100 mg batch had a slightly higher density than the 40 mg or 120 mg batches, leading to a slightly lower Carr Index. The higher density of the 100 mg batch is consistent with slightly fewer fines (<106 μm) and more large granules (>450 μm). Overall, however, the densities and Carr Index are considered similar and reflect acceptable flow for dry granulation tablet formulations. The tablet weight variation during 100 mg batch compression (<1% RSD) and the low USP <905> Acceptance Value (AV=2.7) support good flow properties at multi-kg batch size.

TABLE 5
Powder properties for Development
and GMP blends and granulations.

| | | | | Carr Index |
|---|---|---|---|---|
| Lot No. | Sample | Bulk p (g/cc) | Tap p (g/cc) | (%) |
| 40 mg & 120 | Granulation | 0.45 | 0.59 | 24 |
| mg | | | | |
| 100 mg | Granulation | 0.48 | 0.61 | 21 |
| 40 mg & 120 | Final Blend | 0.44 | 0.59 | 25 |
| mg | | | | |
| 100 mg | Final Blend | 0.46 | 0.60 | 23 |

### Granulation Particle Size Distribution

The particle size distribution of the 40 mg and 120 mg batch granulation are compared to the distributions of the 100 mg batch granulation and final blend in Table 6. The 100 mg batch had somewhat less particles <106 μm, and more large granules, 425-850 μm, than the 40 mg or 120 mg batches.

TABLE 6
Particle size distribution by sieve analysis.

| | 40 mg & 120 mg | | | | | |
|---|---|---|---|---|---|---|
| Particle | Granulation | | 100 mg Granulation | | 100 mg Final Blend | |
| Size (μm) | % Retained | % Cumulative | % Retained | % Cumulative | % Retained | % Cumulative |
| <53/63* | 23.2 | 23.2 | 26.6 | 26.6 | 28.4 | 28.4 |
| 53/63- | 24.3 | 47.5 | 10.1 | 36.7 | 18.6 | 47.0 |
| 106* | | | | | | |
| 106-212 | 13.0 | 60.6 | 9.0 | 45.7 | 9.8 | 56.8 |
| 212-425 | 13.3 | 73.9 | 11.6 | 57.3 | 9.8 | 66.6 |
| 425-850 | 20.6 | 94.5 | 35.2 | 92.5 | 27.5 | 94.1 |
| >850 | 5.5 | 100.0 | 7.5 | 100.0 | 5.9 | 100.0 |

*53 μm screen (#270 mesh) used for the pilot batch, and 63 μm (#230 mesh) was used for the GMP batch.

### Example 5. Tablet Physical Properties

Physical characterization of the 40 mg, 100 mg, and 120 mg batches is summarized below in Table 7. Tablet weight variation was similar for the 40 mg, 100 mg, and 120 mg tablets. The tensile strength, which is calculated from tablet geometry and tablet hardness, of the 100 mg tablets was slightly higher than that of the 40 mg and 120 mg tablet; the 100 mg tablet batch tensile strength was 2.52 MPa, while the 40 mg batch was 2.21 MPa and the 120 mg batch was 2.22 MPa. Additionally, and related, the 100 mg batch solid fraction was 0.80, higher than the values measured for the 120 mg batch, 0.75, and the 40 mg development batch, 0.76. The higher solid fraction is due to tablet thickness being less than the 100 mg development batch. The disintegration time of the 100 mg batch was higher (˜4.8-7.2 min) than the 40 mg or 120 mg batches (˜1.5-2.4 min) and is discussed in the subsequent section in the context of Compressibility, Tabletability, and Compatibility (CTC) profiling.

TABLE 7
Physical characterization and disintegration of main collection
tablets from the 40 mg, 100 mg, and 120 mg batches.

| Parameter | 40 mg | 120 mg | 100 mg |
|---|---|---|---|
| Sample size | n = 20 | n = 20 | n = means from |
| | | | IPT (33 × 5) |
| Shape/Size | SRC 8.73 mm | Mod Cap 9.27 × | SRC 12.00 mm |
| | | 19.00 mm | |
| Mass (mg) [RSD] | 238.3 ± 2.8 [1.2] | 718.0 ± 3.5 [0.5] | 602.2 ± 3.9 [0.7] |
| Hardness (kP) | 12.3 ± 1.0 | 21.1 ± 1.5 | 25.3 ± 1.6 |
| Thickness (mm) | 4.56 ± 0.03 | 5.51 ± 0.03 | 5.79 ± 0.03 |
| Tensile Strength | 2.21 ± 0.17 | 2.22 ± 0.17 | 2.52 ± 0.17 |
| (MPa) | | | |
| Solid Fraction | 0.77 ± 0.01 | 0.75 ± 0.01 | 0.80 ± 0.01 |
| Disintegration | 1:30-2:00 | 1:30-2:18 | 4:44-7:18 |
| Time (first-last, | | | |
| min:s) (n = 3) | | | |
| Disintegration | N/A | N/A | Eroded from the |
| Observations | | | surface to the |
| | | | center of the |
| | | | core; resulting in |
| | | | a donut-like |
| | | | shape, and then |
| | | | thinned and |
| | | | broke into small |
| | | | pieces |

### Example 6. CTC Profiling and Tablet Disintegration

Tablet tensile strength, solid fraction, and disintegration from the CTC profiling are summarized below in Table 8. The disintegration time increases with increasing solid fraction and tensile strength. The 100 mg batch disintegration times are longer than predicted from the 40 mg and 120 mg CTC profiles. The 100 mg batch compression stress-tensile strength relationship on rotary tablet presses shows modestly higher compression stress (212 MPa) was required to achieve 2.5 MPa tensile strength (100 mg batch) than predicted by the 40 mg and 120 mg CTC profiles (estimated to be 175-185 MPa, see Table 8).

TABLE 8
Comparison of disintegration time, tensile strength, and
solid fraction for tablets from CTC profiling during pilot
manufacture (40 mg, 100 mg, and 120 mg batches).

| | | Tensile | | Disintegration |
|---|---|---|---|---|
| | Compression | Strength | Solid | [First-Last] |
| Sample | Stress (MPa) | (MPa) | Fraction | (min) |
| 40 mg | 105 | 0.95 | 0.71 | 0.33-0.50 |
| | 129 | 1.41 | 0.75 | 0.50-0.66 |
| | 185 | 2.52 | 0.79 | 1.30-2.00 |
| | 266 | 3.32 | 0.81 | 2.70-3.30 |
| 120 mg | 105 | 1.18 | 0.70 | 0.83-0.83 |
| | 133 | 1.77 | 0.73 | 0.83-1.00 |
| | 174 | 2.57 | 0.76 | 1.50-2.00 |
| | 248 | 3.43 | 0.79 | 3.50-4.00 |
| 100 mg | 212 | 2.52 | 0.80 | 4.73-7.30 |

### Example 7. Dissolution

Since the disintegration time of the 100 mg tablets was longer than predicted from the 40 mg and 120 mg batches, a comparison of the dissolution profiles is presented in Table 9.

TABLE 9
Dissolution data tables for 40 mg, 100 mg, and 120 mg batch tablets.

| 100 mg | | | 40 mg | | | 120 mg | | |
|---|---|---|---|---|---|---|---|---|
| Time | Amt | Std. | Time | Amt | Std. | Time | Amt | Std. |
| (min) | Dissolved | Dev | (min) | Dissolved | Dev | (min) | Dissolved | Dev |
| 0 | 0.0% | 0.0% | 0 | 0% | 0% | 0% | 0% | 0% |
| 10 | 59.0% | 10.0% | 10 | 65% | 2% | 10% | 71% | 4% |
| 30 | 95.0% | 2.0% | 30 | 74% | 2% | 30% | 86% | 2% |
| 45 | 97.0% | 1.0% | 45 | 89% | 1% | 45% | 92% | 1% |
| 60 | 98.0% | 1.0% | 60 | 95% | 1% | 60% | 93% | 1% |
| 75 | 98.0% | 1.0% | 75 | 96% | 1% | 75% | 71% | 4% |

Those skilled in the art will recognize, or be able to ascertain, using no more than routine experimentation, numerous equivalents to the specific embodiments described specifically herein. Such equivalents are intended to be encompassed in the scope of the following claims.

1. An oral dosage form comprising an intra-granular portion and an extra-granular portion, wherein the intra-granular portion comprises:
about 60.000% w/w to about 70.000% w/w of a solid dispersion comprising a release modifier and Compound A: 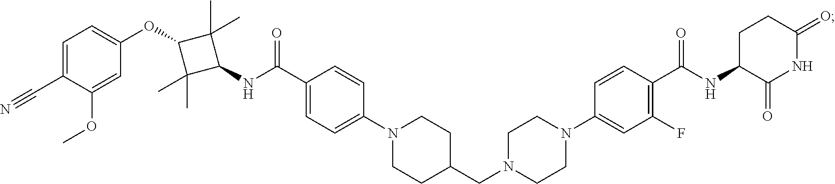 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
a filler;
a disintegrant;
a glidant; and
a lubricant;
and wherein the extra-granular portion comprises:
a disintegrant;
a glidant; and
a lubricant.

2. The oral dosage form of claim 1, wherein the intra-granular portion comprises a first filler and a second filler.

3. The oral dosage form of claim 2, wherein the first filler is microcrystalline cellulose.

4. The oral dosage form of claim 2, wherein the second filler is mannitol.

5-7. (canceled)

8. The oral dosage form of claim 1, wherein the intra-granular portion comprises a first disintegrant and a second disintegrant.

9. (canceled)

10. The oral dosage form of claim 8, wherein the first disintegrant in the intra-granular portion is croscarmellose sodium.

11. The oral dosage form of claim 8, wherein the second disintegrant in the intra-granular portion is crospovidone.

12. (canceled)

13. The oral dosage form of claim 1, wherein the disintegrant in the extra-granular portion is croscarmellose sodium.

14. The oral dosage form of claim 1, wherein the intra-granular portion comprises a first glidant and a second glidant.

15. The oral dosage form of claim 14, wherein the first glidant in the intra-granular portion is silicon dioxide.

16. The oral dosage form of claim 14, wherein the second glidant in the intra-granular portion is colloidal silicon dioxide.

17. The oral dosage form of claim 1, wherein the glidant in the extra-granular portion is colloidal silicon dioxide.

18. The oral dosage form of claim 1, wherein the lubricant in the intra-granular portion is sodium stearyl fumarate.

19. The oral dosage form of claim 1, wherein the lubricant in the extra-granular portion is sodium stearyl fumarate.

20. (canceled)

21. The oral dosage form of claim 3, wherein the amount of microcrystalline cellulose in the intra-granular portion of the oral dosage form is about 4.830% w/w to about 14.830% w/w.

22. The oral dosage form of claim 4, wherein the amount of mannitol in the intra-granular portion of the oral dosage form is about 4.830% w/w to about 14.830% w/w.

23. The oral dosage form of claim 10, wherein the amount of croscarmellose sodium in the intra-granular portion of the oral dosage form is about 1.000% w/w to about 7.000% w/w.

24. The oral dosage form of claim 11, wherein the amount of crospovidone in the intra-granular portion of the oral dosage form is about 1.000% w/w to about 7.000% w/w.

25. The oral dosage form of claim 13, wherein the amount of croscarmellose sodium in the extra-granular portion of the oral dosage form is about 1.500% w/w to about 5.500% w/w.

26. The oral dosage form of claim 15, wherein the amount of silicon dioxide in the intra-granular portion of the oral dosage form is about 0.573% w/w to about 0.773% w/w.

27. The oral dosage form of claim 16, wherein the amount of colloidal silicon dioxide in the intra-granular portion of the oral dosage form is about 0.250% w/w to about 0.750% w/w.

28. The oral dosage form of claim 17, wherein the amount of colloidal silicon dioxide in the extra-granular portion of the oral dosage form is about 0.250% w/w to about 0.750% w/w.

29. The oral dosage form of claim 18, wherein the amount of sodium stearyl fumarate in the intra-granular portion of the oral dosage form is about 0.500% w/w to about 1.500% w/w.

30. The oral dosage form of claim 19, wherein the amount of sodium stearyl fumarate in the extra-granular portion of the oral dosage form is about 0.250% w/w to about 0.750% w/w.

31-56. (canceled)

57. The oral dosage form of claim 1, wherein the release modifier is hydroxypropyl methylcellulose acetate succinate (HPMCAS-M).

58. The oral dosage form of claim 1, wherein the intra-granular portion comprises:
about 66.667% w/w of a solid dispersion comprising a release modifier that is HPMCAS-M and Compound A: 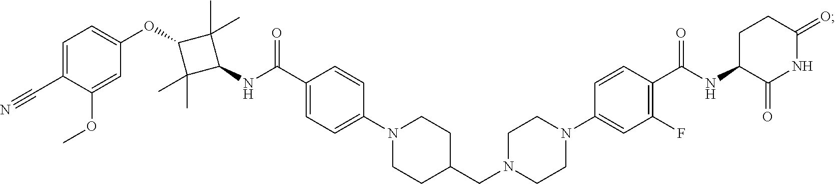 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
and wherein the extra-granular portion comprises:
about 2.500% w/w croscarmellose sodium;
about 0.500% w/w colloidal silicon dioxide; and
about 0.500% w/w sodium stearyl fumarate.

59-68. (canceled)

69. The oral dosage form of claim 1, wherein the oral dosage form is a tablet.

70. (canceled)

71. A method of treating prostate cancer in a subject in need thereof, wherein the method comprises administering a therapeutically effective amount of the dosage form of claim 1 to the subject.

72. (canceled)

73. A combination comprising:
an amorphous form of Compound A: 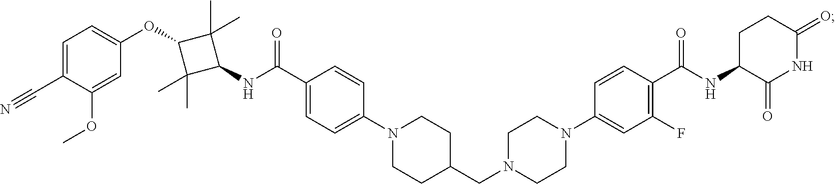 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
and
a release modifier.

74. The combination of claim 73, wherein the release modifier is hydroxypropyl methylcellulose acetate succinate (HPMCAS-M).

75-78. (canceled)

79. The oral dosage form of claim 1, wherein the glidant in the intra-granular portion is colloidal silicon dioxide.

80. The oral dosage form of claim 1, wherein the oral dosage form further comprises a film coat.

81. A tablet selected from tablet 1A, tablet 1D, tablet 1E, and tablet 1I,
wherein:
tablet 1A comprises an intra-granular portion and an extra-granular portion, wherein the intra-granular portion comprises:
about 66.667% w/w of a solid dispersion comprising HPMCAS-M and Compound A in a 1:3 ratio w/w, wherein Compound A is: 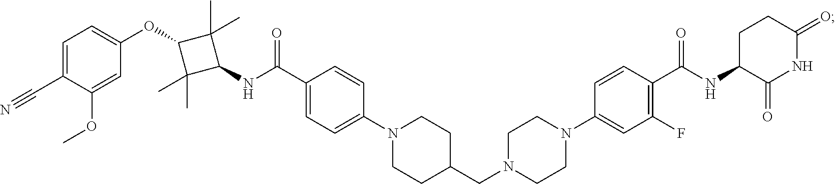 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
about 9.830% w/w microcrystalline cellulose;
about 9.830% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
and wherein the extra-granular portion comprises:
about 2.500% w/w croscarmellose sodium;
about 0.500% w/w colloidal silicon dioxide; and
about 0.500% w/w sodium stearyl fumarate;
tablet 1D comprises an intra-granular portion and an extra-granular portion, wherein the intra-granular portion comprises:
about 66.667% w/w of a solid dispersion comprising HPMCAS-M and Compound A in a 1:3 ratio w/w;
about 9.830% w/w microcrystalline cellulose;
about 9.830% w/w mannitol;
about 4.000% w/w croscarmellose sodium;
about 4.000% w/w crospovidone;
about 1.173% w/w colloidal silicon dioxide; and
about 1.000% w/w sodium stearyl fumarate;
and wherein the extra-granular portion comprises:
about 2.500% w/w croscarmellose sodium;
about 0.500% w/w colloidal silicon dioxide; and
about 0.500% w/w sodium stearyl fumarate;
tablet 1E comprises an intra-granular portion, an extra-granular portion, and a film coat, wherein the intra-granular portion comprises:
about 64.412% w/w of a solid dispersion comprising HPMCAS-M and Compound A in a 1:3 ratio w/w;
about 9.498% w/w microcrystalline cellulose;
about 9.498% w/w mannitol;
about 3.865% w/w croscarmellose sodium;
about 3.865% w/w crospovidone;
about 1.134% w/w colloidal silicon dioxide; and
about 0.966% w/w sodium stearyl fumarate;
wherein the extra-granular portion comprises:
about 2.415% w/w croscarmellose sodium;
about 0.483% w/w colloidal silicon dioxide; and
about 0.483% w/w sodium stearyl fumarate;
and wherein the film coat comprises:
about 3.382% w/w of a combination of polyvinyl alcohol, titanium dioxide, talcum, macrogol, and ferric oxides;
and tablet 1I comprises an intra-granular portion and an extra-granular portion, wherein the intra-granular portion comprises:
about 66.667% w/w of a spray-dried dispersion comprising HPMCAS-M and Compound A in a 1:1 ratio w/w;
about 10.995% w/w microcrystalline cellulose;
about 3.665% w/w mannitol;
about 4.000% w/w croscarmellose sodium;
about 4.000% w/w crospovidone;
about 0.673% w/w silicon dioxide;
about 0.500% w/w colloidal silicon dioxide; and
about 1.000% w/w sodium stearyl fumarate;
and wherein the extra-granular portion comprises:
about 5.000% w/w microcrystalline cellulose;
about 2.500% w/w croscarmellose sodium;
about 0.500% w/w colloidal silicon dioxide; and
about 0.500% w/w sodium stearyl fumarate.
